(19) United States
(12) Patent Application Publication
(10) Pub. No.: US 2017/0305912 A1
(43) Pub. Date: Oct. 26, 2017

(54) PROCESS FOR THE PREPARATION OF 3-PHENYL/HETEROARYL-6-PHENOXY-8-ALKYLAMINO-IMIDAZO[1,2-B]PYRIDAZINE DERIVATIVES

(71) Applicant: Bayer Pharma Aktiengesellschaft, Berlin (DE)

(72) Inventors: Holger PAULSEN, Hilden (DE); Uwe MÜNSTER, Wülfrath (DE); Nicolas GUIMOND, Wuppertal (DE)

(73) Assignee: Bayer Pharma Aktiengesellschaft, Berlin (DE)

(21) Appl. No.: 15/507,716

(22) PCT Filed: Aug. 28, 2015
(86) PCT No.: PCT/EP2015/069739

§ 371 Date: Feb. 28, 2017

(30) Foreign Application Priority Data
Sep. 1, 2014 (EP) 14183029.9

(51) Int. Cl. C07D 487/04; C07B 41/02

(52) U.S. Cl. CPC C07D 487/04; C07B 41/02

(57) ABSTRACT

A process for the preparation of 3-phenyl/heteroaryl-6-phenoxy-8-alkylamino-imidazo[1,2-b]pyridazine derivatives and intermediates of this process. A crystalline form of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide. The compounds are inhibitors of the Mps-1 kinase (Monopolar Spindle 1 kinase; also known as Tyrosine Threonine Kinase, TTK).

The present invention relates to methods of preparing substituted imidazopyridazine compounds of general formula (I) as described and defined herein, as well as to intermediate compounds useful in the preparation of said compounds.

### BACKGROUND OF THE INVENTION

The present invention relates to methods of preparing substituted imidazopyridazine compounds that inhibit Mps-1 (Monopolar Spindle 1) kinase (also known as Tyrosine Threonine Kinase, UK).

Imidazopyridazine derivates have been found to effectively inhibit Mps-1 kinase. Imidazopyridazine derivates and preparation methods therefore are disclosed e.g. in EP2460805A1 and WO2012/032031A1.

Many compounds disclosed in WO2012/032031A1 were prepared according to the following scheme (see e.g. Examples 253, 254, 256, 257, 258, 259, 260, and 262):

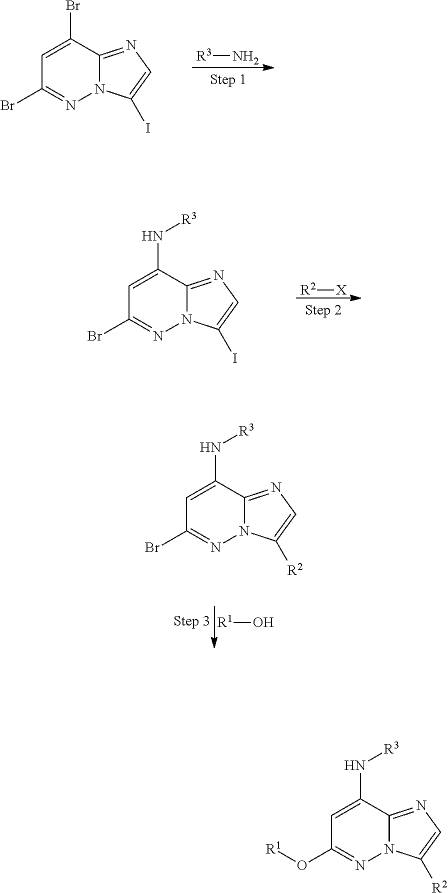

wherein R¹ and R² are optionally substituted phenyl-groups, R³ is an optionally substituted alkyl-group, and X is a boronic acid group or an ester of a boronic acid group.

It turned out that the introduction of the primary amine in Step 1 of the scheme leads to an inactivation of the imidazopyridazine core. The introduction of the hydroxy compound R¹—OH in Step 3 of the scheme has to be performed under comparatively harsh reaction conditions—leading to undesired byproducts and hence an overall yield which has to be increased. Additionally, in case of the preparation process disclosed in WO2012/032031A1, six molar equivalents (which means an excess amount of five mots) of the phenol derivative R¹—OH was required in step 3 to complete the reaction.

### SUMMARY OF THE INVENTION

The present invention provides a method for preparing a compound of general formula (I):

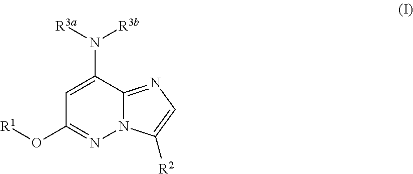

in which
R¹ represents a phenyl- or heteroaryl-group, said phenyl- or heteroaryl-group being optionally substituted, identically or differently, with 1, 2 or 3 substituents selected from: halogen, —CN, C₁-C₃-alkyl-, C₁-C₃-alkoxy-, halo-C₁-C₃-alkyl-, halo-C₁-C₃-alkoxy-;
R² represents a phenyl-group which is optionally substituted, identically or differently, with 1, 2 or 3 substituents selected from: C₁-C₃-alkyl-, —C(═O)N(H)R⁴, —C(═S)N(H)R⁴;
R^{3a} represents a C₁-C₆-alkyl-group, which is optionally substituted, identically or differently, with 1, 2 or 3 substituents selected from: halogen, —CN, C₁-C₃-alkoxy-, halo-C₁-C₃-alkyl-, halo-C₁-C₃-alkoxy-, 3- to 7-membered heterocycloalkyl;
R^{3b} represents hydrogen atom or a C₁-C₆-alkyl-group, which is optionally substituted, identically or differently, with 1, 2 or 3 substituents selected from: halogen, —CN, C₁-C₃-alkoxy-, halo-C₁-C₃-alkyl-, halo-C₁-C₃-alkoxy-, 3- to 7-membered heterocycloalkyl;
R⁴ represents a methy-, ethyl- or cyclopropyl-group; wherein said methyl- or ethyl-group is optionally substituted, identically or differently, with 1, 2, 3 or 4 groups selected from: halogen, —OH, —CN, C₁-C₃-alkoxy-; and wherein said cyclopropyl-group is optionally substituted, identically or differently, with 1, 2, 3 or 4 groups selected from: halogen, —OH, —CN, C₁-C₃-alkoxy-;
the method comprising the following steps:
(a) allowing a compound of general formula (II):

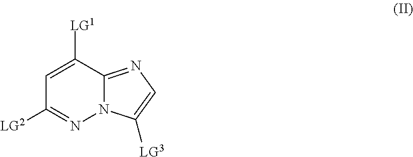

in which LG¹, LG², and LG³ represent leaving groups;
to react with a compound of general formula (III):

R¹—OH (III)

in which R¹ is as defined for general formula (I);
thereby giving a compound of general formula (IV):

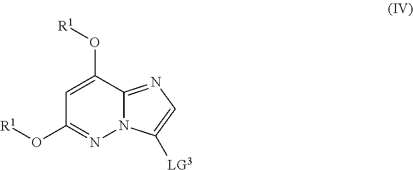

in which R¹ is as defined for general formula (I) and LG³ is as defined for general formula (II);
(b) allowing the compound of general formula (IV):

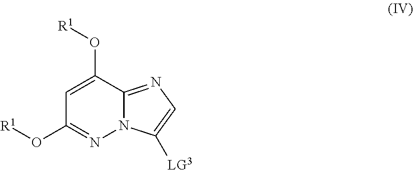

in which R¹ is as defined for general formula (I) and LG³ is as defined for general formula (II);
to react with a compound of general formula (V):

R²—Y (V)

in which R² is as defined for general formula (I) and Y is a group enabling palladium catalysed coupling reactions, including a boronic acid group, an ester of a boronic acid group, a MIDA boronate, and a potassium fluoro borate;
thereby giving a compound of general formula (VI):

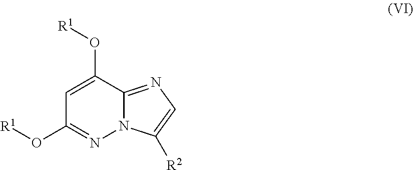

in which R¹ and R² are as defined for general formula (I);
(c) allowing the compound of general formula (VI):

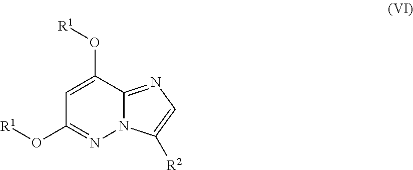

in which R¹ and R² are as defined for general formula (I);
to react with a compound of general formula (VII):

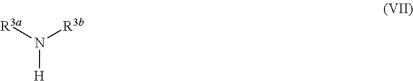

in which R^{3a} and R^{3b} are as defined for general formula (I);
thereby giving a compound of general formula (I).

The present invention is also related to compounds which are used in the preparation of the compounds of general formula (I), supra.

In particular, the present invention covers compounds of general formula (IV):

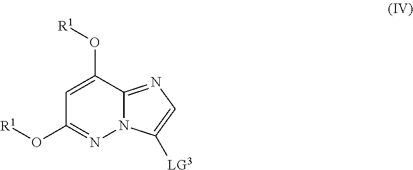

in which R¹ is as defined for general formula (I), supra, and LG³ is a leaving group.

In addition, the present invention covers compounds of general formula (VI):

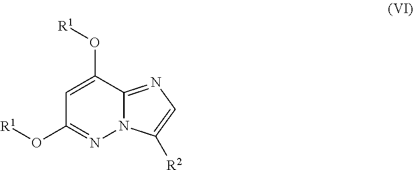

in which R¹ and R² are as defined for general formula (I), supra.

In accordance with yet another aspect, the present invention covers the use of the intermediate compounds of general formula (IV):

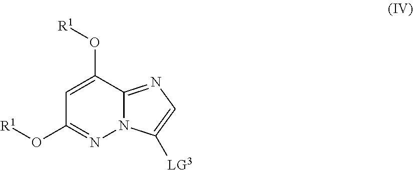

in which R¹ is as defined for general formula (I), supra, and LG³ is a leaving group;
for the preparation of a compound of general formula (I) as defined supra.

In accordance with yet another aspect, the present invention covers the use of the intermediate compounds of general formula (VI):

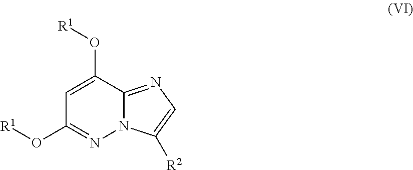

in which R¹ and R² are as defined for general formula (I), supra,
for the preparation of a compound of general formula (I) as defined supra.

In accordance with yet another aspect, the present invention covers a crystalline form of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide obtained as the product of the preparation method according to the present invention, characterized in that the x-ray diffractogram exhibits peak maxima of the 2 theta angle at about 3.7, 17.4, 21.3, and 23.9.

### DETAILED DESCRIPTION OF THE INVENTION

The terms as mentioned in the present text have preferably the following meanings:

The term “halogen atom” or “halo-” is to be understood as meaning a fluorine, chlorine, bromine or iodine atom.

The term “C₁-C₆-alkyl” is to be understood as preferably meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “C₁-C₆-alkylene” is understood as preferably meaning a linear or branched, saturated, divalent hydrocarbon chain (or “tether”) having 1, 2, 3, 4, 5 or 6 carbon atoms, e.g. —CH₂— (“methylene” or “C₁-alkylene”) or, for example —CH₂—CH₂— (“ethylene” or “C₂-alkylene”), —CH₂—CH₂—CH₂—, —C(H)(CH₃)—CH₂— or —C(CH₃)₂—) (“propylene” or “C₃-alkylene”), or, for example —CH₂—C(H)(CH₃)—CH₂—, —CH₂—C(CH₃)₂—), —CH₂—CH₂—CH₂—CH₂— (“butylene” or “C₄-alkylene”), “—C₅-alkylene-”, e.g. —CH₂—CH₂—CH₂—CH₂—CH₂— (“n-pentylene”), or “C₆-alkylene”, e.g. —CH₂—CH₂—CH₂—CH₂—CH₂—CH₂— (“n-hexylene”) group. Particularly, said alkylene tether has 1, 2, 3, 4, or 5 carbon atoms (“C₁-C₅-alkylene”), more particularly 1 or 2 carbon atoms (“C₁-C₂-alkylene”), or, 3, 4, or 5 carbon atoms (“C₃-C₅-alkylene”).

The term “halo-C₁-C₃-alkyl” is to be understood as preferably meaning a linear or branched, saturated, monovalent hydrocarbon group in which the term “C₁-C₃-alkyl” is defined supra, and in which one or more hydrogen atoms is replaced by a halogen atom, in identically or differently, i.e. one halogen atom being independent from another. Particularly, said halogen atom is F. Said halo-C₁-C₃-alkyl group is, for example, —CF₃, —CHF₂, —CH₂F, —CF₂CF₃, —CH₂CF₃ or —CH₂CH₂CF₃.

The term “C₁-C₃-alkoxy” is to be understood as preferably meaning a linear or branched, saturated, monovalent, hydrocarbon group of formula —O—(C₁-C₃-alkyl), in which the term “C₁-C₃-alkyl” is defined supra, e.g. a methoxy, ethoxy, n-propoxy, or iso-propoxy group.

The term “halo-C₁-C₃-alkoxy” is to be understood as preferably meaning a linear or branched, saturated, monovalent C₁-C₃-alkoxy group, as defined supra, in which one or more of the hydrogen atoms is replaced, in identically or differently, by a halogen atom. Particularly, said halogen atom is F. Said halo-C₁-C₃-alkoxy group is, for example, —OCF₃, —OCHF₂, —OCH₂F, —OCF₂CF₃ or —OCH₂CF₃.

The term “3- to 7-membered heterocycloalkyl”, is to be understood as meaning a saturated, monovalent, monocyclic hydrocarbon ring which contains 2, 3, 4, 5 or 6 carbon atoms, and one or more heteroatom-containing groups selected from —C(═O)—, —O—, —S—, —S(═O)—, —S(═O)₂—, —N(R^{a})—, in which R^{a} represents a hydrogen atom or a C₁-C₃-alkyl group; it being possible for said heterocycloalkyl group to be attached to the rest of the molecule via any one of the carbon atoms or, if present, the nitrogen atom.

The term “C₁-C₆”, as used throughout this text, e.g. in the context of the definition of “C₁-C₆-alkyl”, is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, C₁-C₆; particularly C₁-C₂, C₁-C₃, C₁-C₄, C₁-C₅, C₁-C₆; more particularly C₁-C₄; in the case of “C₁-C₃-alkoxy-”, “halo-C₁-C₃-alkyl-” or “halo-C₁-C₃-alkoxy-” even more particularly C₁-C₂.

The term “heteroaryl” is understood as preferably meaning a monovalent, monocyclic-aromatic ring system having 5 or 6 ring atoms, and which contains at least one heteroatom which may be identical or different, said heteroatom being such as oxygen, nitrogen or sulfur. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., or pyridinyl, pyridazinyl, pyrimidinyl, pyrazinyl, triazinyl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridyl includes pyridin-2-yl, pyridin-3-yl, and pyridin-4-yl; or the term thienyl includes thien-2-yl and thien-3-yl. Preferably, the heteroaryl group is a pyridinyl group.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, and (4-methoxy-benzene)sulfonyloxy.

The compounds and intermediates produced according to the steps (a), (b), and (c) may require purification. Purification of organic compounds is well known to the person skilled in the art and there may be several ways of purifying the same compound. In some cases, no purification may be necessary. In some cases, the compounds may be purified by crystallisation. In some cases the compounds may be precipitated from solution by adding an anti-solvent or being added to an anti-solvent. The anti-solvent, e.g. water, may contain additives, e.g. N-acetyl cysteine, as scavenger for Palladium. In some cases, the compounds may be purified by chromatography, particularly flash chromatography, using for example pre-packed silica gel cartridges, e.g. from Separtis such as Isolute® Flash silica gel (silica gel chromatography) or Isolute® Flash NH2 silica gel (aminophase-silica-gel chromatography) in combination with a suitable chromatographic system such as a Flashmaster II (Separtis) or an Isolera system (Biotage) and eluents such as, for example, gradients of hexane/ethyl acetate or DCM/methanol. In some cases, the compounds may be purified by preparative HPLC using, for example, a Waters autopurifier equipped with a diode array detector and/or on-line electrospray ionisation mass spectrometer in combination with a suitable pre-packed reverse phase column and eluants such as, for example, gradients of water and acetonitrile which may contain additives such as trifluoroacetic acid, formic acid or aqueous ammonia.

Generally, the progress of the reactions of steps (a), (b), and (c) can be monitored by removing aliquots from the reactor and analyzing by suitable methods such as thin layer chromatography (TLC), gas chromatography (GC), liquid chromatography (LC) or high performance liquid chromatography (HPLC), or a combination of GC/mass spectroscopy (MS), LC/MS, among other known techniques.

In accordance with a first aspect, the present invention relates to a method for preparing a compound of general formula (I):

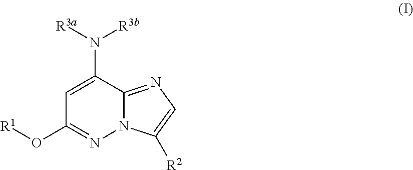

R¹ represents a phenyl- or heteroaryl-group, said phenyl- or heteroaryl-group being optionally substituted, identically or differently, with 1, 2 or 3 substituents selected from: halogen, —CN, C₁-C₃-alkyl-, C₁-C₃-alkoxy-, halo-C₁-C₃-alkyl-, halo-C₁-C₃-alkoxy-.

In a preferred embodiment, R¹ represents a phenyl-group which is optionally substituted, identically or differently, with 1, 2 or 3 substituents selected from: halogen, C₁-C₃-alkyl-, C₁-C₃-alkoxy-, halo-C₁-C₃-alkyl-, halo-C₁-C₃-alkoxy-.

In another preferred embodiment, R¹ represents a phenyl-group which is optionally substituted, identically or differently, with 1, 2 or 3 substituents selected from: halogen, C₁-C₂-alkyl-, C₁-C₂-alkoxy-, halo-C₁-C₂-alkyl-, halo-C₁-C₂-alkoxy-.

In another preferred embodiment, R¹ represents a phenyl-group which is optionally substituted, identically or differently, with 1, 2 or 3 substituents selected from: fluorine, C₁-C₂-alkyl-, C₁-C₂-alkoxy-, fluoro-C₁-C₂-alkyl-, fluoro-C₁-C₂-alkoxy-.

In another preferred embodiment, R¹ represents a phenyl-group which is substituted, identically or differently, with 1, 2 or 3 substituents selected from: fluorine, methoxy-.

In another preferred embodiment, R¹ represents a group selected from:

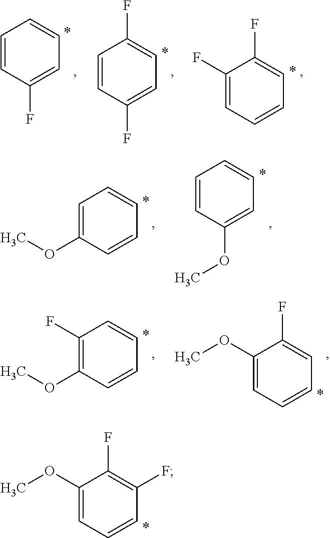

wherein * indicates the point of attachment of said groups to the rest of the molecule.

In another preferred embodiment, R¹ represents a group selected from:

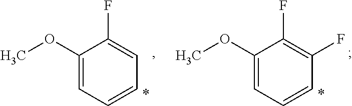

wherein * indicates the point of attachment of said groups to the rest of the molecule.

In another preferred embodiment, R¹ represents:

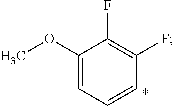

wherein * indicates the point of attachment of said groups to the rest of the molecule.
R² represents a phenyl-group which is optionally substituted, identically or differently, with 1, 2 or 3 substituents selected from: C₁-C₃-alkyl-, —C(═O)N(H)R⁴, —C(═S)N(H)R⁴.

In a preferred embodiment, R² is selected from:

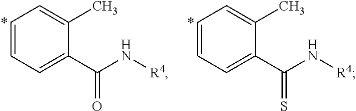

wherein * indicates the point of attachment of said groups to the rest of the molecule.

In another preferred embodiment, R² is selected from:

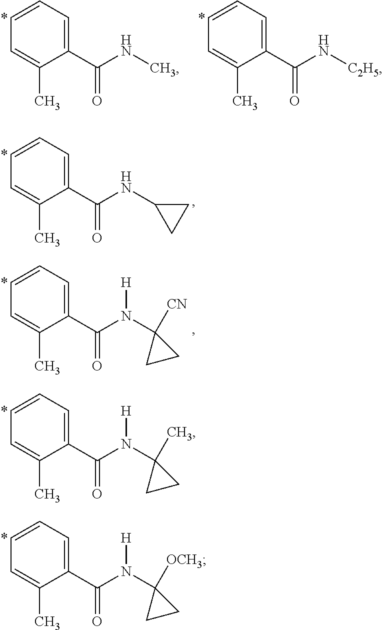

wherein * indicates the point of attachment of said groups with the rest of the molecule.

In another preferred embodiment, R² represents:

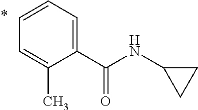

wherein * indicates the point of attachment of said group with the rest of the molecule.

R^{3a} represents a C₁-C₆-alkyl-group, which is optionally substituted, identically or differently, with 1, 2 or 3 substituents selected from: halogen, —CN, C₁-C₃-alkoxy-, halo-C₁-C₃-alkyl-, halo-C₁-C₃-alkoxy-, 3- to 7-membered heterocycloalkyl.

In a preferred embodiment, R^{3a} represents a group selected from:

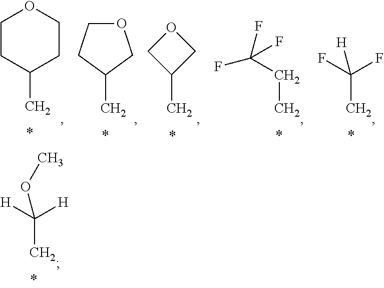

wherein * indicates the point of attachment of said groups with the rest of the molecule.

In another preferred embodiment, R^{3a} represents a group selected from:

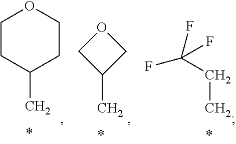

wherein * indicates the point of attachment of said group with the rest of the molecule.

In another preferred embodiment, R^{3a} represents

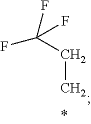

wherein * indicates the point of attachment of said group with the rest of the molecule.
R^{3b} represents hydrogen atom or a C₁-C₆-alkyl-group, which is optionally substituted, identically or differently, with 1, 2 or 3 substituents selected from: halogen, —CN, C₁-C₃-alkoxy-, halo-C₁-C₃-alkyl-, halo-C₁-C₃-alkoxy-, 3- to 7-membered heterocycloalkyl.

In a preferred embodiment, R^{3b} represents hydrogen atom or a C₁-C₆-alkyl-group, which is optionally substituted, identically or differently, with 1, 2 or 3 substituents selected from: halogen, —CN, C₁-C₃-alkoxy-, halo-C₁-C₃-alkyl-, halo-C₁-C₃-alkoxy-.

In another preferred embodiment, R^{3b} represents hydrogen atom.

R⁴ represents a methy-, ethyl- or cyclopropyl-group; wherein said methyl- or ethyl-group is optionally substituted, identically or differently, with 1, 2, 3 or 4 groups selected from: halo-, —OH, —CN, C₁-C₃-alkoxy-; wherein the cyclopropyl-group is optionally substituted, identically or differently, with 1, 2, 3 or 4 groups selected from: halo-, —OH, —CN, C₁-C₃-alkoxy-.

In a preferred embodiment, R⁴ is selected from: methyl-, ethyl-, cyclopropyl-.

In another preferred embodiment, R⁴ represents cyclopropyl-.

The method of the present invention comprises a step (a), in which a compound of general formula (II):

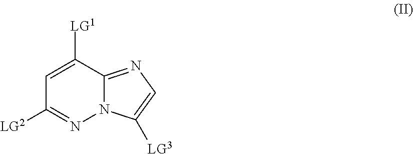

in which LG¹ represents a leaving group, LG² represents a leaving group, and LG³ represents a leaving group;
is reacted with a compound of general formula (III):

R¹—OH (III)

in which R¹ is as defined supra;
thereby giving a compound of general formula (IV):

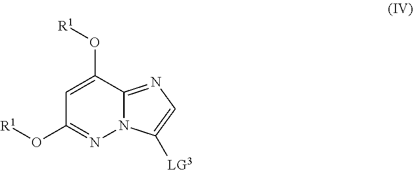

The reaction of the compound of formula (II) with the compound of formula (III) usually is a two step process in which usually the leaving group LG¹ is substituted by the R¹—O— moiety first:

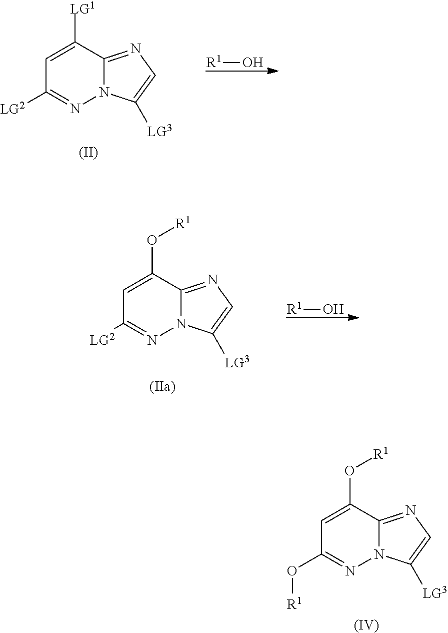

Preferably, LG¹ is selected from: fluoro-, chloro-, bromo-, iodo-, trifluoromethanesulfonyloxy-, p-toluenesulfonyloxy-, and methanesulfonyloxy-.

In a more preferred embodiment, LG¹ represents a bromine atom.

Preferably, LG² is selected from: fluoro-, chloro-, bromo-, iodo-, trifluoromethanesulfonyloxy-, p-toluenesulfonyloxy-, and methanesulfonyloxy-.

In a more preferred embodiment, LG² represents a bromine atom or a chlorine atom.

Preferably, LG³ represents a iodine atom or a bromine atom.

In a more preferred embodiment, LG³ represents a iodine atom.

In an even more preferred embodiment, LG¹ represents a bromine atom, LG² represents a bromine atom or a chlorine atom, and LG³ represents a iodine atom.

Compounds of formula (II) and (III) may be commercially available or can be synthesized according to procedures known to persons skilled in the art, for example applying procedures described in WO2007/38314A2, WO2012/032031A1, EP2460805, and/or WO2014/80633A1.

The coupling of a compound of formula (II) with a compound of formula (III) in principle can be accomplished by a nucleophilic aromatic substitution reaction, in a suitable solvent, such as for example N-methylpyrrolidinone (NMP), dimethylsulfoxid (DMSO), acetone, acetonitrile, N,N-dimethylformamide (DMF), N,N-dimethylacetamide (DMA), ethyl acetate, isopropyl acetate, methyl isobutyl ketone (MIBK), tetradydrofuran (THF), 1,4-dioxane, or sulfolane, or mixtures of said solvents, in the presence of a suitable base, like for example cesium carbonate, potassium carbonate or potassium phosphate.

In a preferred embodiment, step (a) is performed in N-methylpyrrolidinone (NMP) as a solvent using cesium carbonate as a base without any further catalyst and without any ligand.

In another preferred embodiment, step (a) is performed in dimethylsulfoxid (DMSO) as a solvent using potassium carbonate or cesium carbonate as a base without any further catalyst and without any ligand.

The conversion from the compound of formula (II) to the compound of formula (IIa) usually occurs already at room temperature.

Preferably, the reaction mixture for the conversion of a compound of formula (II) to a compound of formula (IV) is heated under stirring to an elevated temperature in the range of 40° C. to 110° C.

So, the substitution of -LG¹ and -LG² by R¹—O— is performed under comparatively mild conditions and with a lower amount of the hydroxy compound R¹—OH than in case of the preparation method described in WO2012/032031A1. The corresponding introduction of the —O-aryl or —O-heteroaryl group in examples 253, 254, 256, 257, 258, 259, 260, and 262 of WO2012/032031A1 was performed at much higher temperatures of 120° C. to 130° C. and a huge excess amount of the hydroxy compound R¹—OH was used.

In a preferred embodiment, the reaction in NMP is performed at a temperature in the range from 60° C. to 90° C., preferably in the range from 65° C. to 75° C.

In another preferred embodiment, the reaction in DMSO is performed at a temperature in the range from 80° C. to 120° C., preferably in the range from 95° C. to 105° C.

The coupling of a compound of formula (II) with a compound of formula (III) in principle can be also accomplished by an Ullmann-type coupling reaction in a suitable solvent, such as for example, NMP, DMF, DMA, DMSO, acetonitrile, water, 1,4-dioxane, collidine (in particular 2,4,6-trimethylpyridine or 2,3,5-trimethylpyridine), diglyme, isobutyramide, a mixture of NMP and 1,1,3,3-tetramethylurea, or sulfolane, or mixtures of said solvents, in the presence of a suitable catalyst, such as, for example, a copper based catalyst like copper(II) diacetate, Cu(I) iodide (in combination with n-butylimidazole and Cs₂CO₃ in NMP), CuI (in combination with tetramethylethylenediamine and K₃PO₄ in DMSO) or CuI (in combination with picolinic acid and K₃PO₄ in DMSO) and in the presence of a suitable base, like for example, cesium carbonate. Optionally, suitable ligands like N,N-dimethylglycine or phenyl hydrogen pyrrolidon-2-ylphosphonate can be added.

After completion of the reaction of a compound of formula (II) with a compound of formula (III), the reaction mixture preferably is cooled down to a temperature in the range of 50° C. to 60° C. The work-up is preferably done by adding of tetrahydrofuran (THF) to the—preferably NMP or DMSO containing—reaction mixture. Inorganic salts are dissolved by addition of water which is preferably heated up to the temperature of the reaction mixture (50° C. to 60° C.). Usually the the product precipitates after dissolution of the inorganic salts.

Subsequently THF may be removed by distillation prior to the isolation by filtration with the aim to decrease the product content in the mother liquor.

The work-up with THF leads to a product which can be filtered off very well.

The method of the present invention further comprises a step (b), in which the compound of general formula (IV):

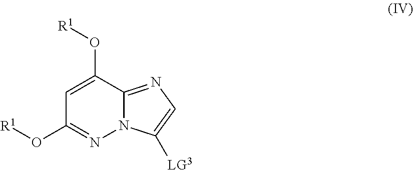

in which R¹ and LG³ are as defined supra;
is reacted with a compound of general formula (V):

R²—Y (V)

in which R² is as defined supra, and Y is a group enabling palladium catalysed coupling reactions, including a boronic acid group, an ester of a boronic acid group, a MIDA boronate, and a potassium fluoro borate;
thereby giving a compound of general formula (VI):

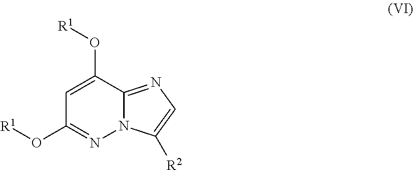

Compounds of formula (V) may be commercially available or can be prepared e.g. from aryl halides [see for example K. L. Billingslay, T. E. Barde, S. L Buchwald, Angew. Chem. 2007, 119, 5455 or T. Graening, Nachrichten aus der Chemie, January 2009, 57, 34]. Examples for the preparation of compounds of formula (V) can also be found e.g. in WO2012/032031A1, EP2460805, and WO2014/80633A1. Compounds of formula (V) may also be prepared in situ from aryl halides and reagents like e.g. tetrahydroxydiboron or bis(pinacolato)diboron and used for Suzuki-coupling without previous isolation.

In a preferred embodiment, R²—Y is selected from:

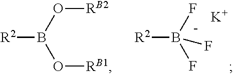

wherein R^{B1} and R^{B2} represent, independently from each other, a hydrogen atom or a C₁-C₆-alkyl- or C₃-C₆-cycloalkyl-group;
or
R^{B1} and R^{B2} together represent a C₁-C₆-alkylene group.

In another preferred embodiment, R²—Y represents an N-methyliminodiacetic acid (MIDA) boronate:

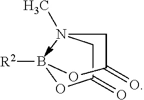

In another preferred embodiment, R²—Y represents

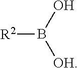

Compounds of formula (IV) can be converted to compounds of general formula (VI) by reaction with R²—Y in the presence of a suitable catalyst system, such as, for example, a palladium based catalyst like, for example, Pd/C, Pd(OH)₂, Palladium (II) acetate, Pd(dba)₂, Pd₂(dba)₃, Pd₂(dba)₃-CHCl₃, Pd(η³-1-PhC₃H₄)(η⁵-C₅H₅) (Organometallics 2012, 31, 2470-2475; J. Org. Chem. 2012, 77, 6908-6916), tetrakis(triphenylphosphine)palladium (0), bis(triphenylphosphine)-palladium (II) chloride or (1,1,-bis(diphenylphosphino)ferrocene)-dichloropalladium (II) and optionally suitable additives such as, for example, phosphines like, for example, P(oTol)₃ or triphenylphosphine and optionally with a suitable base, such as, for example, potassium carbonate, cesium fluoride, cesium carbonate, sodium hydrogen carbonate, tetrabutylammonium fluoride or tribasic potassium phosphate in a suitable solvent, such as, for example acetonitrile, DMF, NMP, 1,4-dioxane, THF, 2-methyltetrahydrofuran, DME, water or mixtures of these solvents at temperatures ranging from room temperature to 100° C., preferably the boiling point of the used solvent.

In a preferred embodiment, step (b) is carried out in a THF/water mixture, the mixture preferably having a THF/water volume quantity ratio in the range from 9:1 to 4:6, using bis(dibenzylideneacetone)palladium(0) as a catalyst, without a phosphine ligand, and with potassium phosphate as a base at a temperature in the range from 60° C. to 80° C., more preferably in the range from 65° C. to 75° C., most preferably at the boiling point of the solution, using a compound of formula (IV) as the educt in which LG³ is a iodine atom. Surprisingly it was found that the Pd(0) catalyzed reaction accomplished without any phosphine ligand leads to a higher yield than the analogous reaction with a phosphine ligand.

In another preferred embodiment, step (b) is carried out in a THF/water mixture, the mixture preferably having a THF/water volume quantity ratio in the range from 9:1 to 4:6, using Pd(η³-1-PhC₃H₄)(η⁵-C₅H₅) as a catalyst and K₃PO₄ a base, at a temperature in the range from 60° C. to 90° C., more preferably in the range from 65° C. to 75° C.

In another preferred embodiment, step (b) is carried out in an acetonitrile/water mixture, the mixture preferably having an acetonitrile/water volume quantity ratio in the range from 2:1 to 1:2, using Pd(η³-1-PhC₃H₄)(η⁵-C₅H₅) as a catalyst and K₂CO₃ a base, at a temperature in the range from 60° C. to 90° C., more preferably in the range from 65° C. to 75° C.

In another preferred embodiment, step (b) is carried out in a THF/water mixture, the mixture preferably having a THF/water volume quantity ratio in the range from 2:1 to 1:2, using dichloro[1,1′-bis(diphenylphoshphino)ferrocene]palladium dichloromethane adduct as a catalyst and K₃PO₄ a base, at a temperature in the range from 60° C. to 90° C., more preferably in the range from 65° C. to 75° C.

After completion of the reaction, N-acetyl cysteine can be added to the reaction mixture in order to remove Pd. Surprisingly it was found that the product of reaction step (b) present in the THF/water mixture is fairly stable against a nucleophilic attack of N-acetyl cysteine.

The crude product can be isolated by filtration of the complete mixture. The product can be cleaned by washing with THF/water mixture, and dissolution in NMP (at a temperature in the range of 55° C. to 75° C.). The amount of residual palladium can be reduced by adding activated carbon and stirring or by filtration of the solution through an activated carbon containing filter. By addition of water or an aqueous solution of N-acetyl-cystein (about 1% by weight, in order to further reduce the Pd content) the cleaned product is precipitated from the solution.

The method of the present invention further comprises a step (c), in which the compound of general formula (VI):

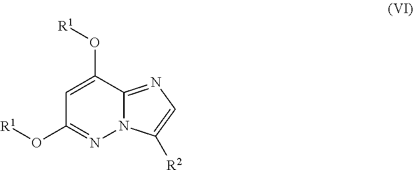

in which R¹ and R² are as defined supra;
is reacted with a compound of general formula (VII):

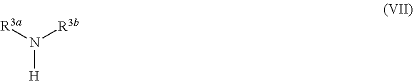

in which R^{3a} and R^{3b} are as defined supra;
thereby giving a compound of general formula (I).

The selective substitution of one of the R¹—O— groups in formula (VI) by a —N(R^{3b})R^{3a} group can be achieved in a suitable solvent such as DMA, N,N-dimethylformamide, DMSO, sulfolane or 1-methylpyrrolidin-2-one, at temperatures ranging from room temperature to the boiling point of the solvent.

In a preferred embodiment, step (c) is carried out in NMP as a solvent, the solvent optionally containing also 1 to 20% by weight of water, at a temperature in the range from 60° C. to 70° C., preferably without any further additive.

In another preferred embodiment, step (c) is carried out in dimethylsulfoxid (DMSO) as a solvent, at a temperature in the range from 90° C. to 110° C., preferably in the range from 95° C. to 105° C., without any further additive.

It was surprisingly found that the substitution is very selective: the substitution takes place at the 8-position of the imidazopyridazine core only.

When the reaction of step (c) is performed in NMP at a temperature of about 65° C. about 6 to 9 equivalents of the amine are needed. When the reaction is performed in DMSO at a temperature in the range from 90° C. to 110° C., 1.5 equivalents of the amine are sufficient in order to completely convert the compound of formula (VI) to the compound of formula (I). In a preferred embodiment, step (c) is performed with 1.3 to 2.5 molar equivalents of the compound of formula (VII) in relation to the amount of the compound of formula (VI), which means 1.3 to 2.5 mots of a compound of formula (VII) are used for the conversion of 1 mol of a compound of formula (VI) to a compound of formula (I).

The product can be isolated by addition of the reaction mixture to water. The precipitated product can be filtered off and cleaned.

In a preferred embodiment, the reaction mixture containing the crude product obtained from step (c) is cooled to a temperature in the range of 45° C. to 55° C. and then is diluted with THF in order to reduce its viscosity. Activated carbon may be used to remove Pd residues.

It is to be understood that the present invention relates also to any combination of the preferred embodiments described herein.

More particularly still, the present invention covers methods of preparation of the compounds of general formula (I) which are disclosed in the Example section of this text, infra.

In a preferred embodiment, the present invention relates to a method for the preparation of a compound selected from:
N-cyclopropyl-4-{6-(3-fluoro-4-methoxyphenoxy)-8-[(oxetan-3-ylmethyl)amino]imidazo[1,2-b]pyridazin-3-yl}-2-methylbenzamide (compound (A)),
N-cyclopropyl-4-{6-(2,3-difluoro-4-methoxyphenoxy)-8-[(3,3,3-trifluoropropyl)amino]imidazo[1,2-b]pyridazin-3-yl}-2-methylbenzamide (compound (B), and
N-cyclopropyl-4-{6-(2,3-difluoro-4-methoxyphenoxy)-8-[(tetrahydro-2H-pyran-4-ylmethyl)amino]imidazo[1,2-b]pyridazin-3-yl}-2-methylbenzamide (compound (C)).

As described in WO 2014/131739, compounds (A), (B) and (C) surprisingly exhibit a superior overall profile with respect to Mps-1-kinase related inhibitory activity in a functional assay (Spindle Assembly Checkpoint Assay), antiproliferative activity (Proliferation Assay with HeLa cells), metabolic stability (in vitro metabolic stability in rat hepatocytes) and drug-drug interaction potential (inhibition of liver enzyme CYP3A4).

In yet another preferred embodiment, the present invention relates to a method for the preparation of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide, the method comprising the following steps:
(a) allowing 8-bromo-6-chloro-3-iodoimidazo[1,2-b]pyridazine or 6,8-dibromo-3-iodoimidazo[1,2-b]pyridazine to react with 2,3-difluoro-4-methoxyphenol; thereby giving 6,8-bis(2,3-difluoro-4-methoxyphenoxy)-3-iodoimidazo[1,2-b]pyridazine;
(b) allowing 6,8-bis(2,3-difluoro-4-methoxyphenoxy)-3-iodoimidazo[1,2-b]pyridazine to react with [4-(cyclopropylcarbamoyl)-3-methylphenyl]boronic acid; thereby giving 4-[6,8-bis(2,3-difluoro-4-methoxyphenoxy)imidazo[1,2-b]pyridazin-3-yl]-N-cyclopropyl-2-methylbenzamide;
(c) allowing 4-[6,8-bis(2,3-difluoro-4-methoxyphenoxy)imidazo[1,2-b]pyridazin-3-yl]-N-cyclopropyl-2-methylbenzamide to react with 3,3,3-trifluoropropan-1-amine; thereby giving N-Cyclopropyl-4-{6-(2,3-difluor-4-methoxyphenoxy)-8-[(3,3,3-trifluorpropyl)amino]imidazo[1,2-b]pyridazin-3-yl}-2-methylbenzamide.

In a preferred embodiment, step (a) is performed in N-methylpyrrolidinone (NMP) as a solvent, using cesium carbonate as a base, at a temperature in the range from 60° C. to 90° C., more preferably in the range from 65° C. to 75° C.

In another preferred embodiment, step (a) is performed in DMSO as a solvent, using potassium carbonate or cesium carbonate as a base, at a temperature in the range from 60° C. to 100° C., more preferably in the range from 65° C. to 75° C.

In a preferred embodiment, step (b) is carried out in a THF/water mixture using bis(dibenzylideneacetone)palladium(0) as a catalyst and potassium phosphate as a base, at a temperature in the range from 60° C. to 80° C., more preferably in the range from 65° C. to 75° C.

In another preferred embodiment, step (b) is carried out in a THF/water mixture using Pd(η³-1-PhC₃H₄)(η⁵-C₅H₅) as a catalyst and K₃PO₄ a base, at a temperature in the range from 60° C. to 90° C., more preferably in the range from 65° C. to 75° C.

In another preferred embodiment, step (b) is carried out in an acetonitrile/water mixture, using Pd(η³-1-PhC₃H₄)(η⁵-C₅H₅) as a catalyst and K₂CO₃ a base, at a temperature in the range from 60° C. to 90° C., more preferably in the range from 65° C. to 75° C.

In another preferred embodiment, step (b) is carried out in a THF/water mixture, using dichloro[1,1′-bis(diphenylphoshphino)ferrocene]palladium dichloromethane adduct as a catalyst and K₃PO₄ a base, at a temperature in the range from 60° C. to 90° C., more preferably in the range from 65° C. to 75° C.

Preferably, step (c) is carried out in dimethylsulfoxid (DMSO) as a solvent, at a temperature in the range from 90° C. to 110° C., preferably in the range from 95° C. to 105° C., without any further additive.

Surprisingly it was found, that a crystalline form of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide can be obtained by adding the reaction mixture obtained from step (c) to water in order to precipitate the product, then—after optionally drying the precipitated product—suspending the precipitated product in toluene, heating the suspension to the boiling point of the suspension in order to azeotropically remove residual water, and then cooling the suspension to a temperature below 50° C., preferably to a temperature in the range of 19° C. to 26° C.

Alternatively, the reaction mixture obtained from step (c) is diluted with THF and then water is added. THF is distilled off, and the precipitated product is filtered off. Then the product is suspended in toluene, the suspension is heated to the boiling point of the suspension in order to azeotropically remove residual water, and then the suspension is cooled to a temperature below 50° C., preferably to a temperature in the range of 19° C. to 26° C.

So, in yet another preferred embodiment, the method as described above further comprises the following steps:

(d) adding the product N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide obtained in step (c) to water in order to precipitate the product;
(e) optionally drying the precipitated product obtained in step (d) in vacuum;
(f) suspending the precipitated product obtained in step (d) or (e) in toluene and heating the suspension to the boiling point of the suspension in order to azeotropically remove residual water;
(g) cooling the suspension obtained in step (f) to a temperature below 50° C., preferably to a temperature in the range from 19° C. to 26° C.; thereby obtaining N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide in crystalline form.

The crystalline form of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide is easily filterable; the loss of material which remains in the toluene mother liquid is negligible.

WO 2014/131739 describes a method for the preparation of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide resulting in amorphous material. The crystalline form of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide as obtained from the process as described above has not been disclosed so far.

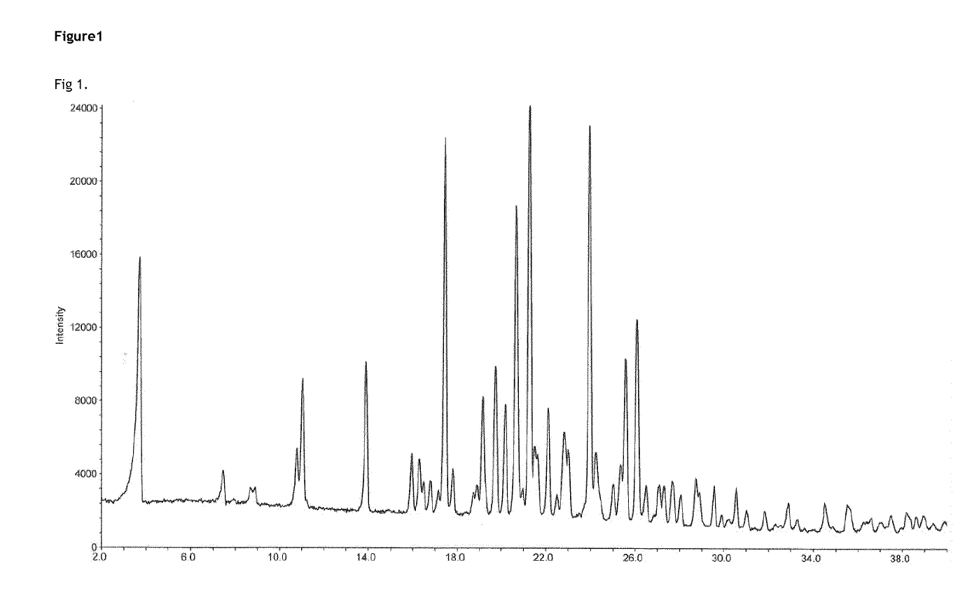

FIG. 1 shows the x-ray diffractogram of the crystalline form of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide. Table 1 lists the corresponding powder diffraction data (strongest reflections):

TABLE 1
X-ray powder diffraction data - strongest reflections

| | 2Theta |
|---|---|
| D (Å) | (°) |
| 23.51 | 3.7 |
| 8.20 | 10.8 |
| 8.00 | 11.1 |
| 6.36 | 13.9 |
| 5.56 | 15.9 |
| 5.44 | 16.3 |
| 5.08 | 17.4 |
| 4.63 | 19.1 |
| 4.50 | 19.7 |
| 4.40 | 20.2 |
| 4.29 | 20.7 |
| 4.17 | 21.3 |
| 4.13 | 21.5 |
| 4.02 | 22.1 |
| 3.89 | 22.8 |
| 3.86 | 23.0 |
| 3.71 | 23.9 |
| 3.67 | 24.2 |
| 3.51 | 25.3 |
| 3.48 | 25.6 |
| 3.42 | 26.1 |
| 3.11 | 28.7 |

Data collection for X-ray powder diffraction (XRPD) was carried out in transmission mode on automated STOE Powder Diffractometers using germanium-monochromatized Cuκα₁-radiation. The X-ray tube with copper anode was operated by 40 kV and 40 mA. The 2Θ scans were performed between 2°≦2Θ40° 2Θ35° (stepwidth 0.5°). Data acquisition and evaluation were performed using the STOE WinX^{pow} software package.

The present invention further provides N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide in crystalline form, characterized in that the x-ray diffractogram exhibits peak maxima of the 2 theta angle at about 3.7, 17.4, 21.3, and 23.9.

One of ordinary skill in the art will appreciate that an X-ray diffraction pattern may be obtained with a measurement error that is dependent upon the measurement conditions employed. In particular, it is generally known that intensities in an X-ray diffraction pattern may fluctuate depending upon crystal habitus of the material and measurement conditions employed. It is further understood that relative intensities may also vary depending upon experimental conditions and, accordingly, the exact order of intensity should not be taken into account. Additionally, a measurement error of diffraction angle theta for a conventional X-ray diffraction pattern at a given temperature is typically about ±0.1, and such degree of measurement error should be taken into account as pertaining to the aforementioned diffraction angles. Consequently, the term “about” when used herein in reference to X-ray powder diffraction patterns means that the crystal forms of the instant invention are not limited to the crystal forms that provide X-ray diffraction patterns completely identical to the X-ray diffraction patterns depicted in the accompanying FIGURE disclosed herein. Any crystal form that provides X-ray diffraction patterns that is substantially identical to those disclosed in the accompanying FIGURE falls within the scope of the present invention. The ability to ascertain whether the polymorphic forms of a compound are the same albeit the X-ray diffraction patterns are not completely identical is within the purview of one of ordinary skill in the art.

It turned out that the crystalline form of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide prepared by the method of the present invention comprising steps (a) to (g), supra, is characterized by an advantageous particle size distribution. Table 2 shows the particle size distribution of 6 different batches of N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide represented by the X90, X50, and X10 values. The particle size distribution was determined in a dry dispersion using the device “Sympatec Helos” and the method “AM-PE 69” (Pharmdoss).

Batch Nos. 1 to 5 were prepared by the method of the present invention comprising steps (a) to (c), supra. In case of Batch Nos. 2 to 5, the preparation method comprised the additional steps (d) to (g) (in case of Batch No. 1 steps (d) to (g) were not applied).

It can be clearly seen that the particles of Batch Nos. 2 to 5 are smaller than the particles of Batch No. 1 and the distribution is more uniform. In case of Batch Nos. 2 to 5, further micronization of the particles might not be necessary in order to increase the solubility or bioavailability of the final drug.

TABLE 2
Particle Size Distribution

| Batch No. | X90 [μm] | X50 [μm] | X10 [μm] |
|---|---|---|---|
| 1 | 249.0 | 18.2 | 2.0 |
| 2 | 36.1 | 5.3 | 1.4 |
| 3 | 9.9 | 4.1 | 1.2 |
| 4 | 13.2 | 4.2 | 1.3 |
| 5 | 12.4 | 4.0 | 1.2 |

X90 = particle diameter corresponding to 10% of the cumulative undersize distribution by volume, μm.
X10 = particle diameter corresponding to 90% of the cumulative undersize distribution by volume, μm.
X50 = particle diameter corresponding to 50% of the cumulative undersize distribution by volume, μm.

In accordance with a further aspect, the present invention covers intermediate compounds which are useful in the preparation of compounds of the present invention of general formula (I), particularly in the method described herein.

In particular, the present invention covers compounds of general formula (IV):

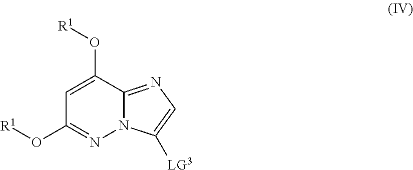

in which R¹ and LG³ are as defined supra.

In a preferred embodiment the compound of general formula (IV) is selected from:

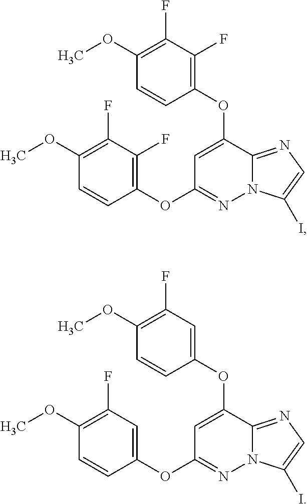

In addition, the present invention covers compounds of general formula (VI):

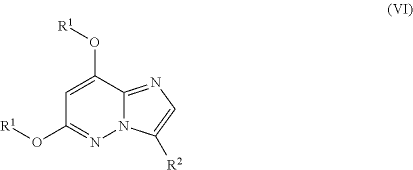

in which R¹ and R² are as defined for general formula (I), supra.

In a preferred embodiment the compound of general formula (VI) is selected from:

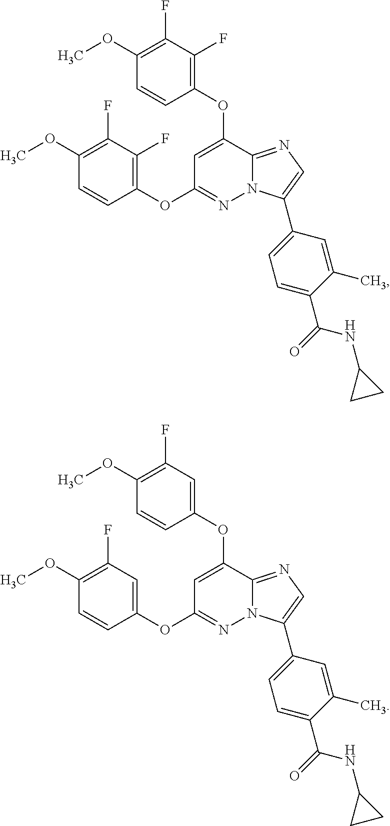

### Experimental Section

### General

The following Table lists the abbreviations used in this paragraph, and in the Examples section.

| Abbreviation | Meaning |
|---|---|
| DMSO | dimethylsulfoxide |
| HPLC | high performance liquid chromatography |
| LC-MS | liquid chromatography - mass spectrometry |
| MW | molecular weight |
| NMP | N-methylpyrrolidinone |
| NMR | nuclear magnetic resonance |
| ppm | parts per million |
| RT | retention time |
| THF | tetrahydrofuran |

### Method A (LC-MS):

Instrument: Waters ACQUITY SQD UPLC System; column: Waters Acquity UPLC HSS T3 1.8μ 50×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow-rate: 0.40 ml/min; UV-detection: 208-400 nm.

### Method B (LC-MS):

Instrument: Waters ACQUITY SQD UPLC System; column: Waters Acquity UPLC HSS T3 1.8μ 50×1 mm; eluent A: 1 l Wasser+0.25 ml 99% ige Ameisensäure, Eluent B: 1 l acetonitrile+0.25 ml 99% ige Ameisensäure; gradient: 0.0 min 95% A→6.0 min 5% A→7.5 min 5% A oven: 50° C.; flow-rate: 0.35 ml/min; UV-detection: 210-400 nm.

### Method C (LC-MS): TOF-M2

Instruments: MS: Waters Synapt G2S; UPLC: Waters Acquity I-CLASS; column: Waters, HSST3, 2.1×50 mm, C18 1.8 μm; eluent A: 1 l water+0.01% formic acid; eluent B: 1 l acetonitrile+0.01% formic acid; gradient: 0.0 min 2% B→2.0 min 2% B→13.0 min 90% B→15.0 min 90% B; oven: 50° C.; flow-rate: 1.20 ml/min; UV-detection: 210 nm

### Method D (LC-MS): MCW-LTQ-POROSHELL-TFA98-10 min

Instruments: MS: ThermoFisherScientific LTQ-Orbitrap-XL; Instruments HPLC: Agilent 1200SL; column: Agilent, POROSHELL 120, 3×150 mm, SB—C18 2.7 μm; Eluent A: 1 l Wasser+0.1% trifluoroacetic acid; Eluent B: 1 l acetonitrile+0.1% trifluoroacetic acid; Gradient: 0.0 min 2% B→0.3 min 2% B→5.0 min 95% B→10.0 min 95% B; oven: 40° C.; flow-rate: 0.75 ml/min; UV-detection: 210 nm.

### Example 1

### 6,8-Bis(2,3-difluoro-4-methoxyphenoxy)-3-iodoimidazo[1,2-b]pyridazine

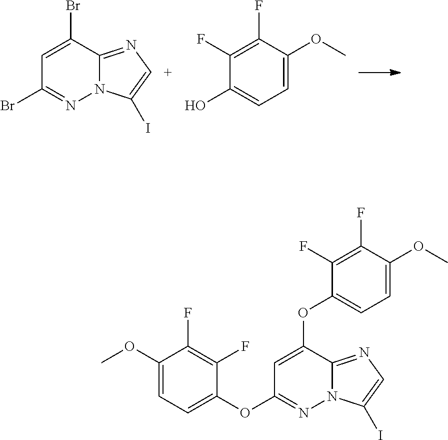

1.00 kg of 6,8-dibromo-3-iodoimidazo[1,2-b]pyridazine (2.48 mol), 0.87 kg of 2,3-difluoro-4-methoxyphenol (5.46 mol) and 2.43 kg of cesium carbonate (7.45 mol) were stirred in 5.0 L NMP and heated to 70° C. After 4 h at 70° C. the reaction mixture was cooled to 50-60° C. and 5.0 L of THF were added. 20.0 L of water were heated to 55° C. and added to the suspension within 12 min. After dissolution of the inorganic salts the product precipitated from clear solution. The temperature was raised to approx. 87° C. and approx. 4 L solvent (mainly THF) were removed by distillation. The mixture was cooled to 20-22° C. within 2 h and stirred at this temperature for 14 h. The product was isolated by suction filtration, rinsed two times with water (2.0 L each) and dried in vacuum at 40° C. for 20 h to mass constance. 1.38 kg (99%) of the title compound were obtained as a slightly grey solid.

¹H-NMR (DMSO-d6): δ=3.91 (3H), 3.94 (3H), 6.58 (1H), 7.06-7.21 (2H), 7.24-7.32 (1H), 7.35-7.43 (1H), 7.76 (1H) ppm.

LC-MS (Method A): RT=1.21 min; m/z (ES+) 562.0 g/mol [M+H]⁺; required MW=560.98 (exact mass).

### Example 2

### 6,8-Bis(2,3-difluoro-4-methoxyphenoxy)-3-iodoimidazo[1,2-b]pyridazine

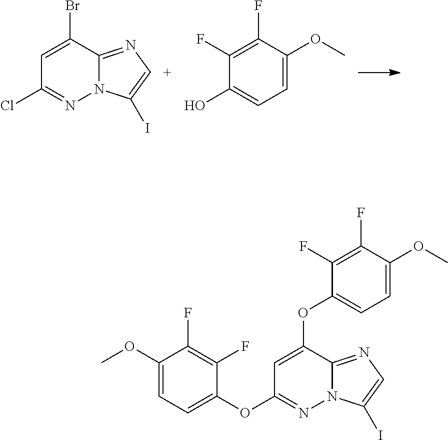

10.0 g of 6-chloro-8-bromo-3-iodoimidazo[1,2-b]pyridazine (27.9 mmol), 9.83 g of 2,3-difluoro-4-methoxyphenol (61.4 mmol) and 11.6 g of potassium carbonate (83.7 mmol) were stirred in 50 mL DMSO and heated to 100° C. After 5 h at 100° C. the reaction mixture was cooled to 50° C. and 50 mL of THF were added. 200 mL of water were added slowly to the suspension at 50° C. After dissolution of the inorganic salts the product precipitated from clear solution. The mixture was heated with a jacket temperature of 100° C. until an internal temperature of approx. 87° C. was reached and 38 mL solvent (mainly THF) had been removed by distillation. The mixture was cooled to 20° C. and stirred at this temperature for 3 h. The product was isolated by suction filtration, rinsed three times with water (5 mL each) and dried in vacuum at 50° C. overnight. 15.6 g (99.6%) of the title compound were obtained in 97% purity (by HPLC).

### Example 3

### 4-[6,8-bis(2,3-difluoro-4-methoxyphenoxy)imidazo[1,2-b]pyridazin-3-yl]-N-cyclopropyl-2-methylbenzamide

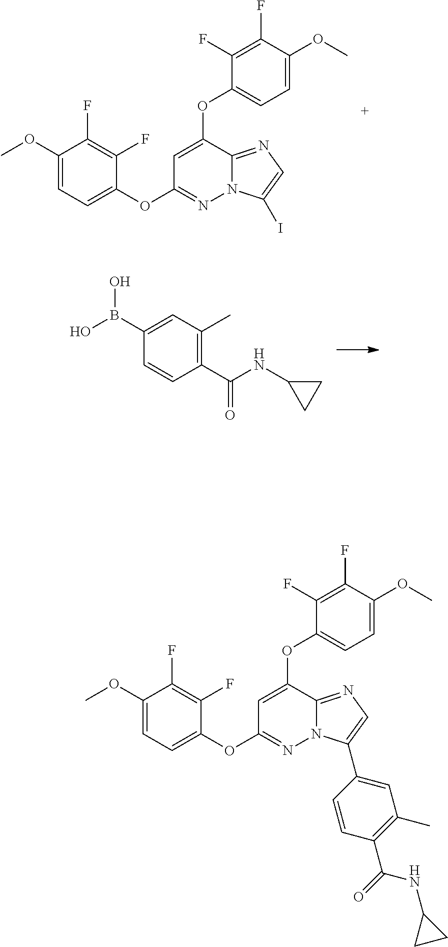

1.5 kg 6,8-Bis(2,3-difluoro-4-methoxyphenoxy)-3-iodoimidazo[1,2-b]pyridazine (2.67 mol) prepared as described in example 1 and 1.14 kg potassium phosphate (5.35 mol) were suspended and stirred in 7.5 L THF and 7.5 L water at room temperature. 0.64 kg of [4-(cyclopropylcarbamoyl)-3-methylphenyl]boronic acid (2.94 mol) were added to the mixture to give a brown suspension. The temperature rose to 30° C. The reaction mixture was inertized three times by evacuating and flushing with nitrogen. To this suspension 15.4 g Bis(dibenzylideneacetone)palladium(0) (26.7 mmol) were added and the reaction vessel was inertized once more. The reaction mixture was heated to 70° C. and stirred at this temperature for 5 h. 0.22 kg N-acetyl cysteine (1.35 mol) were added and stirring was continued for 1 h. After cooling to 20° C. and stirring at this temperature for 30 min a crude product was isolated by filtration of the complete mixture. This crude product was washed two times with 2.88 l water/THF (1:1) each on the filter plate and additional 4 times with 1.44 L water/THF (1:1) each and then dried in vacuum at 50° C. for 17 h. The crude product (1.38 kg) was dissolved in 12.3 L NMP at room temperature and heated to 60° C. The solution was filtered through a preheated filter plate (50° C.) which was rinsed with 1.4 L NMP. To the combined filtrate 2.74 kg of an aqueous solution of N-acetyl cysteine (1 weight-%) was added at 50° C. over 1 h to precipitate the product. The suspension was cooled to room temperature within 3 h and stirred over night. The product was isolated by suction filtration. After washing with water (two times with 2.87 L each), with THF/water 1:1 (2 L) and with water again (2.87 L) the product was dried in vacuum at 50° C. 1.26 kg (78%) of the title compound were obtained as a white to slightly grey powder.

¹H-NMR (DMSO-d6): δ=0.48-0.55 (2H), 0.65-0.72 (2H), 2.14 (3H), 2.78-2.86 (1H), 3.93 (3H), 3.95 (3H), 6.62 (1H), 7.13-7.22 (2H), 7.22-7.26 (1H), 7.30-7.37 (1H), 7.39-7.46 (1H), 7.65-7.70 (1H), 7.72 (1H), 8.22 (1H), 8.28-8.31 (1H) ppm.

LC-MS (Method A): RT=1.16 min; m/z (ES+) 609.2 g/mol [M+H]⁺; required MW=608.17 (exact mass).

### Example 4

### 4-[6,8-bis(2,3-difluoro-4-methoxyphenoxy)imidazo[1,2-b]pyridazin-3-yl]-N-cyclopropyl-2-methylbenzamide

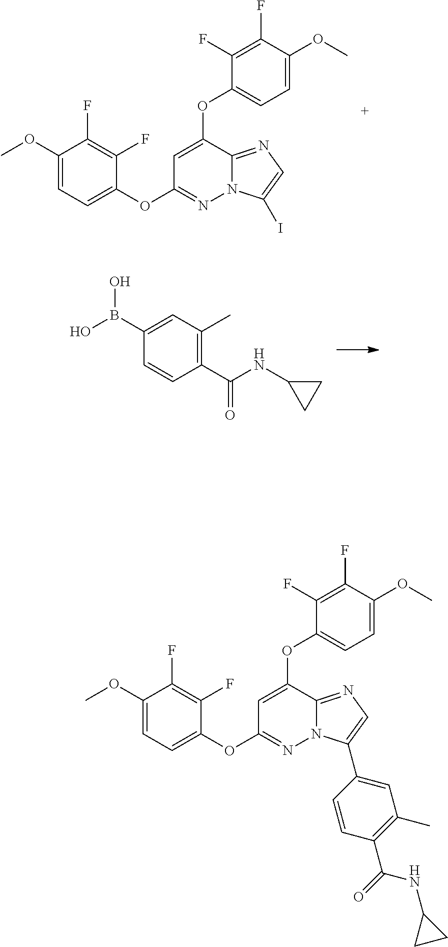

5.0 g of 6,8-Bis(2,3-difluoro-4-methoxyphenoxy)-3-iodoimidazo[1,2-b]pyridazine (8.9 mmol), 2.1 g of [4-(cyclopropylcarbamoyl)-3-methylphenyl]boronic acid (9.8 mmol), 51 mg bis(dibenzylideneacetone)palladium(0) (0.089 mmol) and 2.5 g potassium carbonate (17.8 mmol) were suspended under argon in a degassed mixture of acetonitrile (25 mL) and water (25 mL) at room temperature. The reaction mixture was heated with a jacket temperature of 70° C. and stirred at this temperature for 4 h. 0.73 g N-acetyl cysteine (4.5 mmol) were added and strirring was continued for 1 h. After cooling to 20° C. over 30 min the crude product was isolated by filtration. This crude product was rinsed three times with acetonitrile/water 1:1 (10 ml each) on the filter plate. The residue was dried in vacuum (approx. 60 mbar) at 45° C. overnight. 4.8 g (89%) of the crude product were obtained. 4.3 g were dissolved in 43 ml NMP at 50° C. and 1.2 g of charcoal added and stirred for 15 min. The solution was filtered and the filter rinsed two times with 10 ml NMP each. 20 ml of an aqueous solution containing 1% of N-acetyl cysteine by weight were added to the filtrate at 50° C. over a period of 1 h. The resulting suspension was cooled to 23° C. over 3 h and stirred overnight. The product was filtered, rinsed with water three times (10 ml each) and dried <200 mbar at 45° C. over three days. 3.4 g (63%) of the title compound were obtained.

### Example 5

### 4-[6,8-bis(2,3-difluoro-4-methoxyphenoxy)imidazo[1,2-b]pyridazin-3-yl]-N-cyclopropyl-2-methylbenzamide

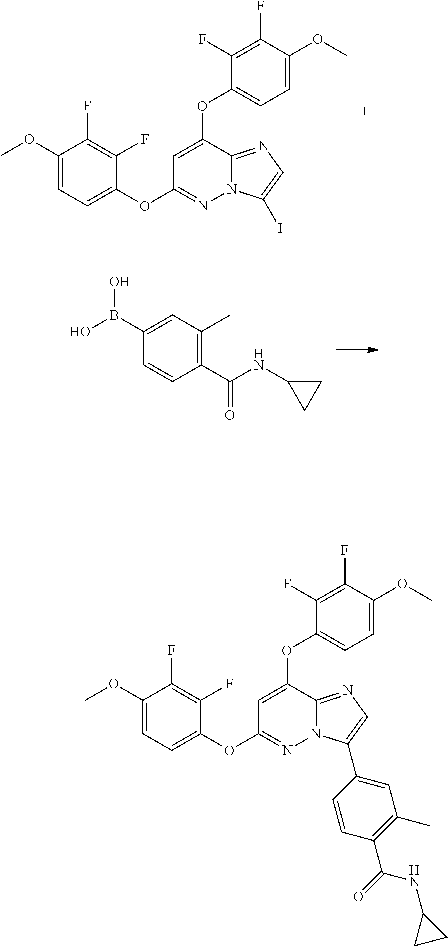

10.0 g of 6,8-Bis(2,3-difluoro-4-methoxyphenoxy)-3-iodoimidazo[1,2-b]pyridazine (17.8 mmol), 4.3 g of [4-(cyclopropylcarbamoyl)-3-methylphenyl]boronic acid (19.6 mmol), 51 mg (0.18 mmol) Pd(η³-1-PhC₃H₄)(η⁵-C₅H₅) and 7.6 g potassium phosphate (35.6 mmol) were suspended under argon in a degassed mixture of THF (50 mL) and water (50 mL) at room temperature. The reaction mixture was heated with a jacket temperature of 70° C. and stirred at this temperature for 6 h. 1.45 g N-acetyl cysteine (8.9 mmol) were added and strirring was continued for 1 h. After cooling to 20° C. over 30 min the crude product was isolated by filtration. This crude product was rinsed three times with water/THF 1:1 (25 ml each) on the filter plate. The residue was dried in vacuum (approx. 60 mbar) at 45° C. overnight. 8.5 g (78%) of the product were obtained with an HPLC purity of 96% by area.

### Example 5a

### 4-[6,8-bis(2,3-difluoro-4-methoxyphenoxy)imidazo[1,2-b]pyridazin-3-yl]-N-cyclopropyl-2-methylbenzamide

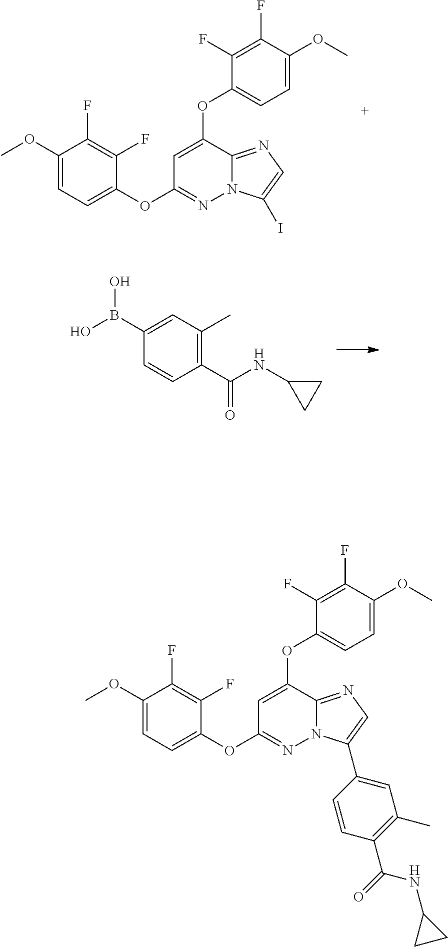

5.0 g of 6,8-Bis(2,3-difluoro-4-methoxyphenoxy)-3-iodoimidazo[1,2-b]pyridazine (8.9 mmol), 2.1 g of [4-(cyclopropylcarbamoyl)-3-methylphenyl]boronic acid (9.8 mmol) and 3.8 g potassium phosphate (17.8 mmol) were suspended under argon in a degassed mixture of THF (25 mL) and water (25 mL) at room temperature. 18 mg dichloro[1,1′-bis(diphenyl-phoshphino)ferrocene]palladium dichloromethane adduct (0.022 mmol) were added and the reaction mixture was heated with a jacket temperature of 70° C. and stirred at this temperature for 10 h followed by stirring at room temperature overnight. 0.73 g N-acetyl cysteine (4.5 mmol) were added after reheating to 70° C. (jacket temperature) and stirring was continued for 1 h. After cooling to 20° C. over 30 min and stirring for 1 h the crude product was isolated by suction filtration. The crude product was rinsed three times with water/THF 1:1 (10 ml each) on the filter plate. The residue was dried in vacuum (approx. 60 mbar) at 45° C. overnight. 4.5 g (83%) of the crude product were obtained with an HPLC purity of 98.5% by area. The product was suspended in NMP (45 ml) and dissolved at 50° C.

An aqueous 1% solution of N-acetylcysteine (9 ml) was added over 1 h. The mixture was cooled to 23° C. over 1 h and stirring was continued at room temperature for 1 h. The precipitate was isolated by suction filtration, washed three times with water (10 ml each) and dried in vacuum at 45° C. overnight. 3.7 g (68%) of the title compound were isolated with an HPLC purity of >99.5% by area.

### Example 6

### 4-[6,8-bis(2,3-difluoro-4-methoxyphenoxy)imidazo[1,2-b]pyridazin-3-yl]-N-cyclopropyl-2-methylbenzamide

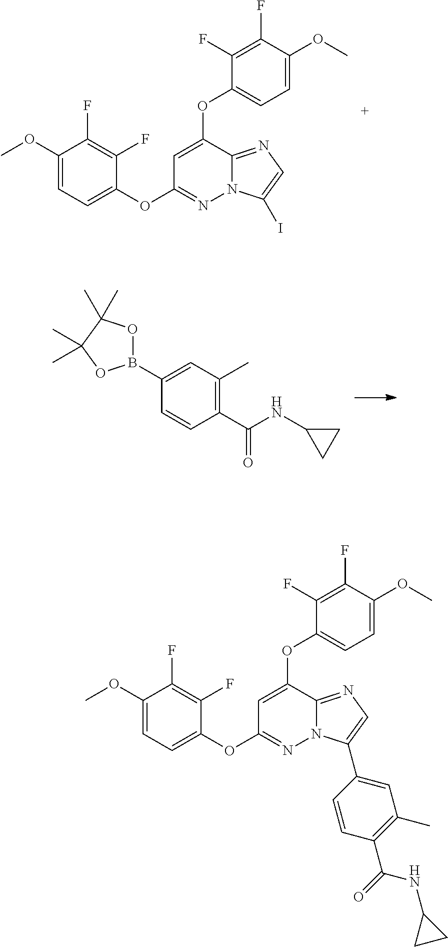

8.5 g 6,8-Bis(2,3-difluoro-4-methoxyphenoxy)-3-iodoimidazo[1,2-b]pyridazine (15.1 mmol), 5.0 g of N-cyclopropyl-2-methyl-4-(4,4,5,5-tetramethyl-1,3,2-dioxaborolan-2-yl)benzamide, 87 mg bis(dibenzylideneacetone)palladium(0) (0.15 mmol) and 6.4 g potassium phosphate (30.2 mmol) were suspended under argon in a degassed mixture of THF (42 mL) and water (42 mL) at room temperature. The reaction mixture was heated with a jacket temperature of 70° C. and stirred at this temperature for 7 h and at room temperature overnight. The mixture was heated to 70° C. again, 2.5 g N-acetyl cysteine (15.1 mmol) were added and strirring was continued for 1 h. After cooling to room temperature the crude product was isolated by suction filtration and rinsed three times with water/THF (1:1) (20 mL each) on the filter plate. The crude product was dissolved in 90 ml NMP at 50° C. 18 mL of water containing 1% of N-acetyl cysteine are added over a period of 1 h. The mixture is cooled to room temperature over a period of 3 h and stirred overnight. The title compound is isolated by suction filtration and rinsing with water (3×, 18 mL each). After drying at 45° C. and approx. 100 mbar 6.8 g (74%) of the product are obtained.

### Example 7

### 4-[6,8-bis(2,3-difluoro-4-methoxyphenoxy)imidazo[1,2-b]pyridazin-3-yl]-N-cyclopropyl-2-methylbenzamide

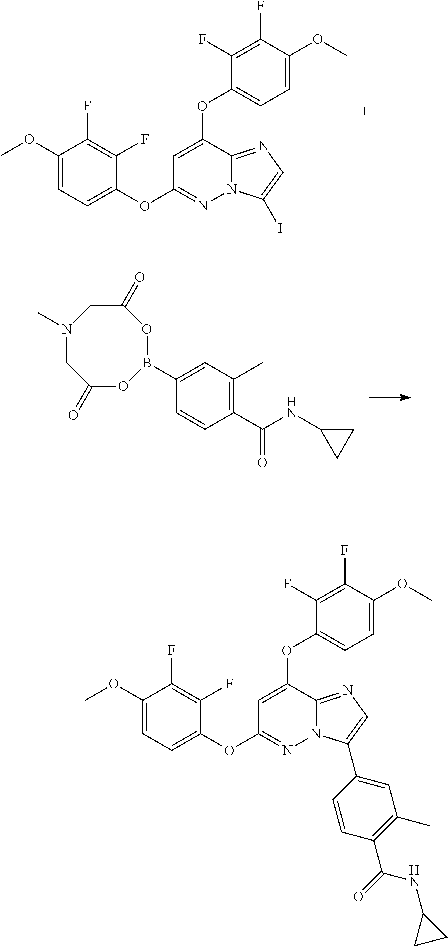

5.0 g 6,8-Bis(2,3-difluoro-4-methoxyphenoxy)-3-iodoimidazo[1,2-b]pyridazine (8.9 mmol), 3.2 g of N-cyclopropyl-2-methyl-4-(6-methyl-4,8-dioxo-1,3,6,2-dioxazaborocan-2-yl)benzamide (9.8 mmol), 51 mg bis(dibenzylideneacetone)palladium(0) (0.089 mmol) and 3.8 g potassium phosphate (17.8 mmol) were suspended under argon in a degassed mixture of THF (25 mL) and water (25 mL) at room temperature. The reaction mixture was heated with a jacket temperature of 70° C. and stirred at this temperature for 23 h. Additional 51 mg catalyst were added and the reaction continued for 2 h. 0.73 g N-Acetylcysteine (4.5 mmol) were added and stirring continued for 1 h. The reaction mixture was cooled to room temperature. Product was isolated by filtration and washing three times with THF/water 1:1 (15 ml each). 2.9 g (53%) of the title compound were isolated with a HPLC-purity of 92% by area.

### Intermediate N-cyclopropyl-2-methyl-4-(6-methyl-4,8-dioxo-1,3,6,2-dioxazaborocan-2-yl)benzamide

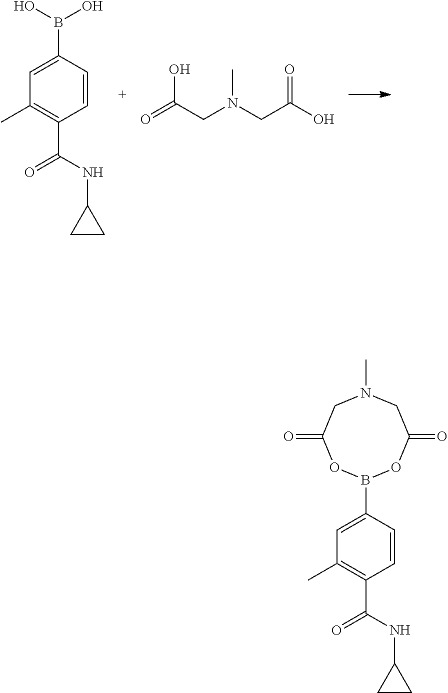

10 g boronic acid (45.7 mmol) and 6.7 g 2,2′-(methylimino)diacetic acid (MIDA) (45.7 mmol) were suspended in 300 ml of toluene and 70 ml of DMSO. The mixture was heated to reflux with a jacket temperature of 120° C. for 18 h. Water was removed with a Dean-Stark-trap. The reaction mixture was cooled to room temperature and 240 ml brine (10 wgt-% aq-solution) and 700 ml ethyl acetate were added. The product precipitated in the aqueous phase. The organic phase was washed two times with 240 ml brine each. The organic phase was dried and evaporated to dryness to give 1.9 g of a mixture containing boronic acid (⅓) and title compound (⅔). The title compound was isolated in 95% HPLC-purity by filtration of the combined aqueous phases. 10.3 g (68%) of the title compound were obtained after drying overnight at 65 mbar and 45° C.

¹H-NMR (DMSO-d6): δ=0.48-0.55 (2H), 0.63-0.70 (2H), 2.51 (3H—overlap with solvent signal), 2.31 (3H), 2.78-2.86 (1H), 4.08-4.16 (2H), 4.30-4.37 (2H), 7.22-7.29 (3H), 8.24 (1H) ppm.

LC-MS (Method D): RT=4.10 min; m/z (ES+) 331.14 g/mol [M+H]⁺; required MW=330.14 (exact mass).

### Example 8

### N-Cyclopropyl-4-{6-(2,3-difluor-4-methoxyphenoxy)-8-[(3,3,3-trifluorpropyl)amino]imidazo[1,2-b]pyridazin-3-yl}-2-methylbenzamide

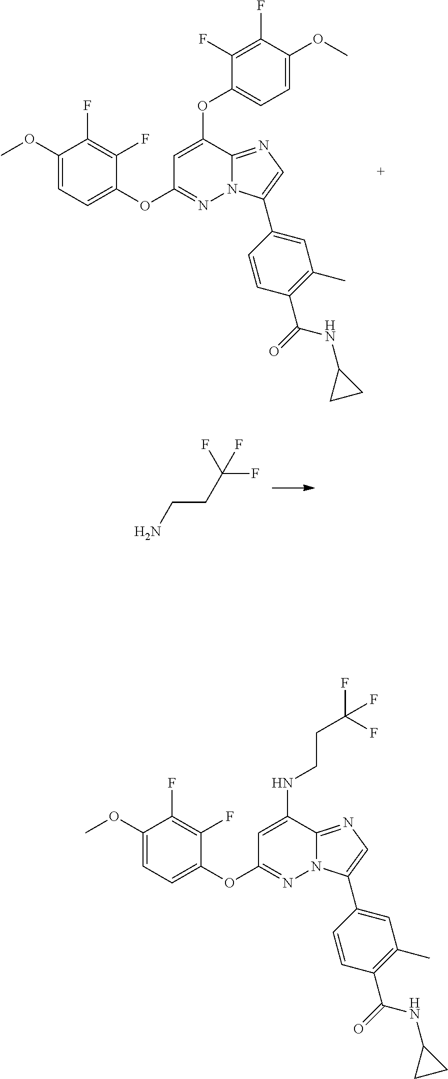 [SMILES: COc1ccc(Oc2cc(Oc3ccc(OC)c(F)c3F)c3ncc(-c4ccc(C(=O)NC5CC5)c(C)c4)n3n2)c(F)c1F ; NCCC(F)(F)F ; COc1ccc(Oc2cc(NCCC(F)(F)F)c3ncc(-c4ccc(C(=O)NC5CC5)c(C)c4)n3n2)c(F)c1F]

Synthesis and isolation of the title compound were performed under containment conditions for highly potent active ingredients and under nitrogen atmosphere:

1.00 kg (1.64 mol) of 4-[6,8-bis(2,3-difluoro-4-methoxyphenoxy)imidazo[1,2-b]pyridazin-3-yl]-N-cyclopropyl-2-methylbenzamide prepared as described in example 3 and 0.37 kg of trifluoropropaneamine (3.29 mol) in 5.5 kg of dimethylsulfoxide were heated to 100° C. within 50 in. Stirring was continued at this temperature for 20 h. The reaction mixture was cooled to 50° C. and filtered through a filter plate and a 2 μm steel filter cartridge. The filtered solution was directly added to 13.8 kg of water at 5° C.-28° C. over a period of 8 h to precipitate the product. Rinsing was performed with 0.7 kg DMSO. The aqueous mixture was heated to 80° C. for approx. 2:45 h then cooled to 25° C. and stirred at room temperature for approx. 12 h. The crude product was isolated by filtration and washed with water (4.3 L each). The product was dried in vacuum (end vacuum 27 mbar) at 50-55° C. for approx. 22:40 h. The crude product was suspended in 10.5 kg toluene and heated to 110° C. with azeotropic removal of residual water (if present). After 3 h at reflux the mixture was cooled to 20-25° C. and the crystalline product was isolated by filtration and washing with toluene three times (1 kg each). After drying in vacuum with nitrogen flushing at 45-55° C. for 22:30 h (end vacuum 3 mbar) 0.82 kg (88%) of a white solid were obtained.

### Example 9

### N-Cyclopropyl-4-{6-(2,3-difluor-4-methoxyphenoxy)-8-[(3,3,3-trifluorpropyl)amino]imidazo[1,2-b]pyridazin-3-yl}-2-methylbenzamide

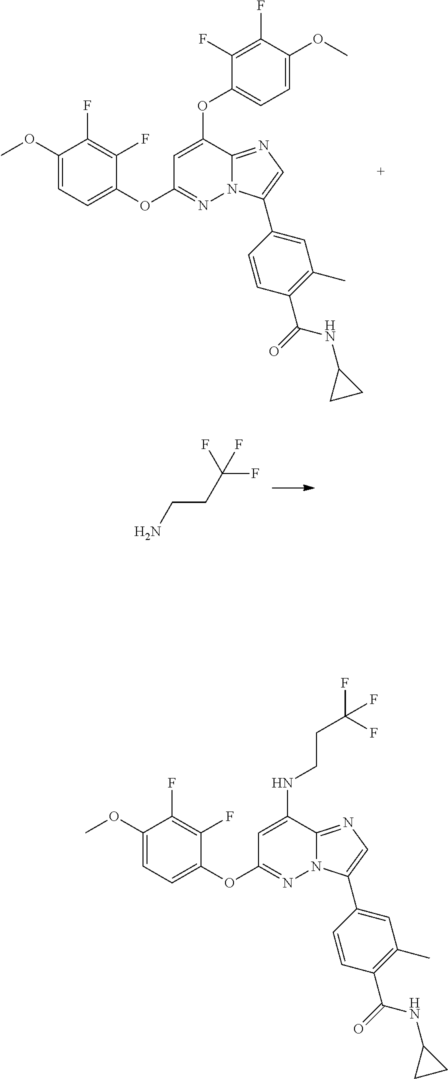 [SMILES: COc1ccc(Oc2cc(Oc3ccc(OC)c(F)c3F)c3ncc(-c4ccc(C(=O)NC5CC5)c(C)c4)n3n2)c(F)c1F ; NCCC(F)(F)F ; COc1ccc(Oc2cc(NCCC(F)(F)F)c3ncc(-c4ccc(C(=O)NC5CC5)c(C)c4)n3n2)c(F)c1F]

50.0 g (82.2 mmol) of 4-[6,8-bis(2,3-difluoro-4-methoxyphenoxy)imidazo[1,2-b]pyridazin-3-yl]-N-cyclopropyl-2-methylbenzamide prepared as described in example 3 and 18.6 g of trifluoropropaneamine (164.3 mmol) in 250 ml of dimethylsulfoxide were heated to 100° C. Stirring was continued at this temperature for 20 h. The reaction mixture was cooled to 50° C. and diluted with 250 ml of THF. 1 g of charcoal (Norit A Supra®) was added and the mixture stirred at 50° C. for 30 min. After filtration (Seitz filter plate K100) and rinsing with 20 ml THF water (610 ml) was added to the filtrate over 30 min at 50° C. giving a thin suspension. The mixture was heated with a mantle temperature of 120° C., distilling of approx. 240 ml solvent. During distillation the temperature of the mixture rose from 65° C. to 89° C. After cooling to room temperature the suspension was stirred overnight. The precipitate was isolated by suction filtration and washed 4× with water (100 ml each). The crude product was suspended in toluene (610 ml) and heated with a mantle temperature of 140° C. with azeotropic removal of water (approx. 25 ml) until the final temperature of the mixture reached 108° C. The mixture was cooled to room temperature and stirred for 30 min. The product was isolated by suction filtration and rinsed 3× with toluene (60 ml each). Drying in vacuum at 40° C. yielded 43.1 g (93%) of the title compound.

¹H-NMR (DMSO-d6+D₂O): δ=0.47-0.53 (2H), 0.63-0.72 (2H), 2.10 (3H), 2.63-2.75 (2H), 2.79 (1H), 3.65 (2H), 3.90 (3H), 6.20 (1H), 7.10 (1H), 7.19 (1H), 7.24 (1H), 7.61 (1H), 7.70 (1H), 7.94 (1H) ppm.

The two exchangeable N—H-protons at 7.75-7.81 ppm (1H) and 8.24 ppm (1H) (compare: PCT/EP2014/053573) were suppressed by proton-exchange with D₂O.

LC-MS (Method A): RT=1.16 min; m/z (ES+) 562.3 g/mol [M+H]⁺; required MW=561.18.

### Example 10

### 6,8-Bis(2,3-difluoro-4-methoxyphenoxy)-3-bromoimidazo[1,2-b]pyridazine

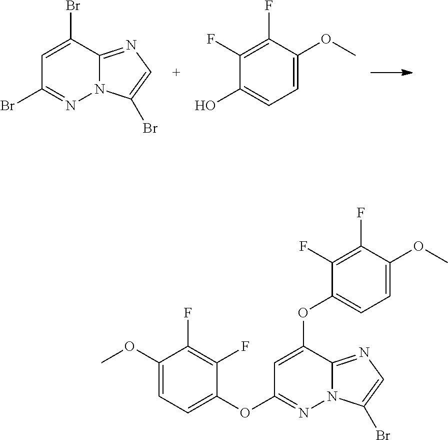

3.30 g of 3,6,8-tribromo-3-iodoimidazo[1,2-b]pyridazine (9.27 mmol), 3.27 g of 2,3-difluoro-4-methoxyphenol (20.40 mmol) and 9.07 g of cesium carbonate (27.82 mmol) were stirred in 33 mL NMP and heated to 60° C. After 19 h at 60° C. the reaction mixture was cooled to 50° C. and 33 mL of THF were added. The suspension was added to 165 mL water at room temperature. THF (26 mL) was removed by distillation and the suspension was cooled to 20° C. After stirring at 20° C. for 2 h the product was isolated by suction filtration and washed with water three times (25 ml each). The product was dried over night in vacuum at 45° C. 4.4 g (92% of theory) of the title compound were obtained.

¹H-NMR (DMSO-d6): δ=3.91 (3H), 3.94 (3H), 6.61 (1H), 7.06-7.14 (1H), 7.14-7.21 (1H), 7.25-7.32 (1H), 7.36-7.43 (1H), 7.80 (1H) ppm.

LC-MS (Method B): RT=3.86 min; m/z (ES+) 514.0 g/mol [M+H]+; required MW=512.99 (exact mass).

### Example 11

### 6,8-Bis(2,3-difluoro-4-ethoxyphenoxy)-3-iodoimidazo[1,2-b]pyridazine

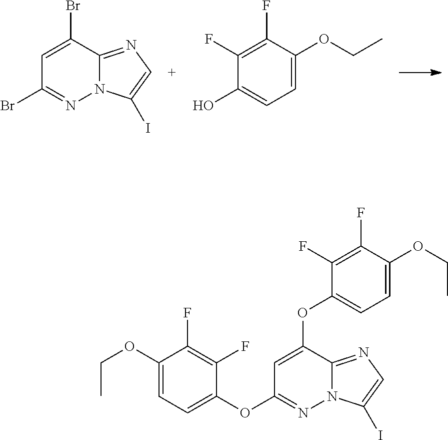 [SMILES: Brc1cc(Br)c2ncc(I)n2n1 ; CCOc1ccc(O)c(F)c1F ; CCOc1ccc(Oc2cc(Oc3ccc(OCC)c(F)c3F)c3ncc(I)n3n2)c(F)c1F]

12 g of 6,8-dibromo-3-iodoimidazo[1,2-b]pyridazine (29.8), 11.4 g of 2,3-difluoro-4-ethoxy-phenol (65.5 mmol) and 29.1 g of cesium carbonate (89.4 mmol) were stirred in 60 mL NMP and heated to 70° C. After 2 h at 70° C. the reaction mixture was cooled to 50° C. and 60 mL of THF were added. At 50° C. 240 mL of water were added slowly to the suspension. The mixture was heated with a jacket temperature of 100° C. until an internal temperature of approx. 87° C. was reached and 46 mL solvent (mainly THF) had been removed by distillation. The mixture was cooled to 20° C. and stirred at this temperature overnight. The product was isolated by suction filtration, rinsed three times with water (12 mL each) and dried in vacuum at 40° C. overnight. 16.6 g (95%) of the title compound were obtained.

¹H-NMR (DMSO-d6): δ=1.35-1.43 (6H), 4.14-4.24 (4H), 6.57 (1H), 7.05-7.19 (2H), 7.21-7.30 (1H), 7.32-7.41 (1H), 7.76 (1H) ppm.

LC-MS (Method C): RT=9.91 min; m/z (ES+) 590.0 g/mol [M+H]⁺; required MW=589.01 (exact mass).

### Example 12

### 4-[6,8-bis(2,3-difluoro-4-ethoxyphenoxy)imidazo[1,2-b]pyridazin-3-yl]-N-cyclopropyl-2-methylbenzamide

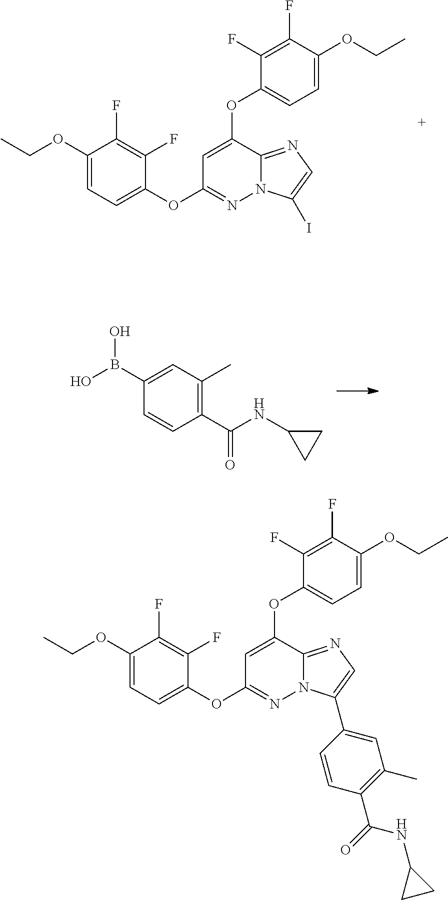

10.0 g of 6,8-Bis(2,3-difluoro-4-ethoxyphenoxy)-3-iodoimidazo[1,2-b]pyridazine (17.0 mmol), 4.1 g of [4-(cyclopropylcarbamoyl)-3-methylphenyl]boronic acid (18.7 mmol), 98 mg bis(dibenzylideneacetone)palladium(0) (0.17 mmol) and 7.2 g potassium phosphate (33.9 mmol) were suspended under argon in a degassed mixture of THF (50 mL) and water (50 mL) at room temperature. The reaction mixture was heated with a jacket temperature of 70° C. and stirred at this temperature for 9 h. After stirring for 14 h at room temperature the mixture was heated with a jacket temperature of 70° C. again and 1.39 g N-acetyl cysteine (8.5 mmol) were added and strirring was continued for 1 h. After cooling to 23° C. over 30 min the crude product was isolated by filtration. This crude product was rinsed three times with 25 mL water/THF (1:1)—each—on the filter plate. The residue was dried in vacuum (approx. 60 mbar) at 45° C. overnight. 5.4 g (50%) of the title compound was obtained.

¹H-NMR (DMSO-d6): δ=0.48-0.55 (2H), 0.65-0.72 (2H), 1.35-1.45 (6H), 2.15 (3H), 2.78-2.86 (1H), 4.15-4.26 (4H), 6.62 (1H), 7.11-7.21 (2H), 7.21-7.26 (1H), 7.27-7.34 (1H), 7.36-7.44 (1H), 7.65-7.70 (1H), 7.72 (1H), 8.22 (1H), 8.27-8.32 (1H) ppm.

LC-MS (Method D): RT=6.38 min; m/z (ES+) 637.20 g/mol [M+H]⁺; required MW=636.20 (exact mass).

### Example 13

### N-Cyclopropyl-4-{6-(2,3-difluor-4-ethoxyphenoxy)-8-[(3,3,3-trifluorpropyl)amino]imidazo[1,2-b]pyridazin-3-yl}-2-methylbenzamide

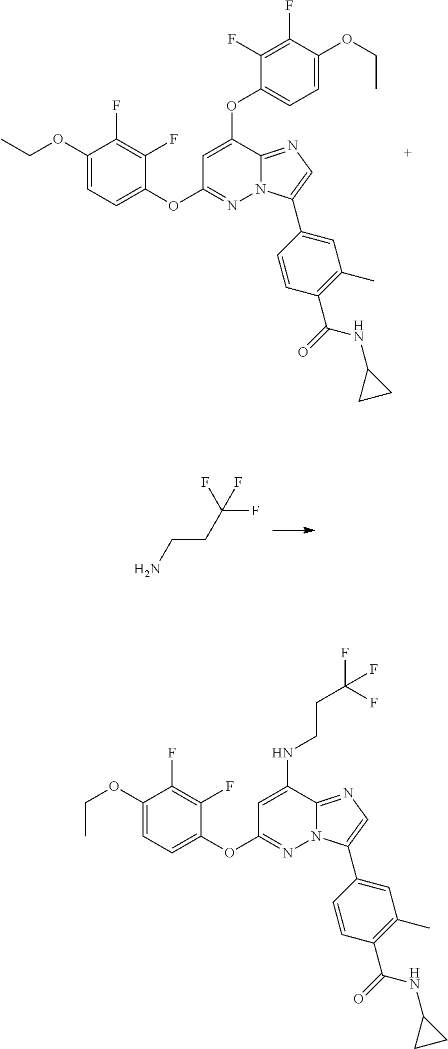 [SMILES: CCOc1ccc(Oc2cc(Oc3ccc(OCC)c(F)c3F)c3ncc(-c4ccc(C(=O)NC5CC5)c(C)c4)n3n2)c(F)c1F ; NCCC(F)(F)F ; CCOc1ccc(Oc2cc(NCCC(F)(F)F)c3ncc(-c4ccc(C(=O)NC5CC5)c(C)c4)n3n2)c(F)c1F]

200 mg of 4-[6,8-bis(2,3-difluoro-4-ethoxyphenoxy)imidazo[1,2-b]pyridazin-3-yl]-N-cyclopropyl-2-methylbenzamide (0.31 mmol) and 71 mg of 3,3,3-trifluoropropanamine (0.63 mmol) were heated in 1 ml DMSO to 100° C. After 20 h of stirring at this temperature the LC-MS analysis showed complete conversion to the title compound and the corresponding release of the ethoxydifluorophenol-leaving group.

LC-MS (Method D): RT=5.97 min; m/z (ES+) 576.20 g/mol [M+H]⁺; required MW=575.20 (exact mass).

1. A method for preparing a compound of general formula (I): 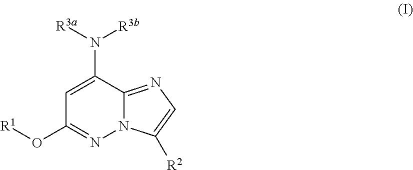
in which:
R¹ represents a phenyl- or heteroaryl-group, said phenyl- or heteroaryl-group being optionally substituted, identically or differently, with 1, 2 or 3 substituents selected from: halogen, —CN, C₁-C₃-alkyl-, C₁-C₃-alkoxy-, halo-C₁-C₃-alkyl-, halo-C₁-C₃-alkoxy-;
R² represents a phenyl-group which is optionally substituted, identically or differently, with 1, 2 or 3 substituents selected from: C₁-C₃-alkyl-, —C(═O)N(H)R⁴, —C(═S)N(H)R⁴;
R^{3a} represents a C₁-C₆-alkyl-group, which is optionally substituted, identically or differently, with 1, 2 or 3 substituents selected from: halogen, —CN, C₁-C₃-alkoxy-, halo-C₁-C₃-alkyl-, halo-C₁-C₃-alkoxy-3- to 7-membered heterocycloalkyl;
R^{3b} represents hydrogen atom or a C₁-C₆-alkyl-group, which is optionally substituted, identically or differently, with 1, 2 or 3 substituents selected from: halogen, —CN, C₁-C₃-alkoxy-, halo-C₁-C₃-alkyl-, halo-C₁-C₃-alkoxy-, 3- to 7-membered heterocycloalkyl;
R⁴ represents a methyl, ethyl- or cyclopropyl-group; wherein said methyl- or ethyl-group is optionally substituted, identically or differently, with 1, 2, 3 or 4 substituents selected from: halogen, —OH, —CN, C₁-C₃-alkoxy-; and wherein the cyclopropyl-group is optionally substituted, identically or differently, with 1, 2, 3 or 4 substituents selected from: halogen, —OH, —CN, C₁-C₃-alkoxy-;
the method comprising the following steps:
(a) allowing a compound of general formula (II): 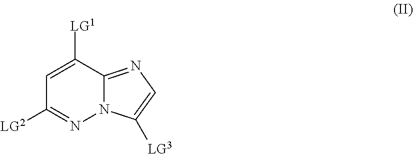
in which LG¹ represents a leaving group;
LG² represents a leaving group;
and LG³ represents a leaving group;
to react with a compound of general formula (III):
R¹—OH (III)
in which R¹ is as defined for the compound of general formula (I);
thereby giving a compound of general formula (IV): 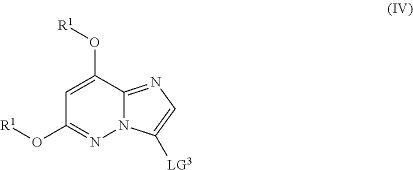
(b) allowing the compound of general formula (IV): 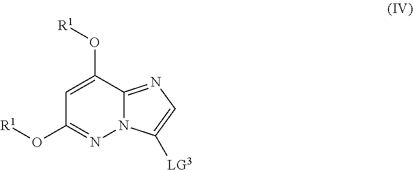
to react with a compound of general formula (V):
R²—Y (V)
in which R² is as defined for the compound of general formula (I) and Y is a group enabling palladium catalysed coupling reactions, including a boronic acid group, an ester of a boronic acid group, a MIDA boronate, and a potassium fluoro borate;
thereby giving a compound of general formula (VI): 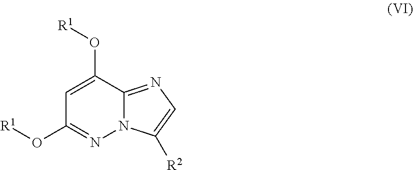
(c) allowing the compound of general formula (VI): 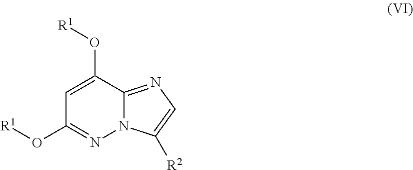
to react with a compound of general formula (VII): 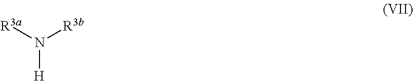
in which R^{3a} and R^{3b} are as defined for the compound of general formula (I);
thereby giving the compound of general formula (I).

2. The method according to claim 1, characterized in that
LG¹ represents a bromine atom;
LG² represents a bromine atom or a chlorine atom; and
LG³ represents a iodine atom.

3. The method according to claim 1, characterized in that step (a) is performed in N-methylpyrrolidinone as a solvent using cesium carbonate as a base without any further catalyst and ligand.

4. The method according to claim 1, characterized in that step (a) is performed in dimethylsulfoxide as a solvent using potassium carbonate or cesium carbonate as a base without any further catalyst and ligand.

5. The method according to claim 2, characterized in that step (b) is carried out in a THF/water mixture, using bis(dibenzylideneacetone)palladium(0) as a catalyst, without a phosphine ligand, and with potassium phosphate as a base.

6. The method according to claim 2, characterized in that step (b) is carried out in a THF/water mixture, using Pd(η³-1-PhC₃H₄)(η⁵-C₅H₅) as a catalyst and K₃PO₄ as a base, at a temperature in the range from 60° C. to 90° C.

7. The method according to claim 2, characterized in that step (b) is carried out in a THF/water mixture, using dichloro[1,1′-bis(diphenylphoshphino)ferrocene]palladium dichloromethane adduct as a catalyst and K₃PO₄ a base, at a temperature in the range from 60° C. to 90° C.

8. The method according to claim 1, characterized in that step (c) is carried out in dimethylsulfoxide using 1.3 to 2.5 molar equivalents of the compound of formula (VII) in relation to the amount of the compound of formula (VI).

9. The method according to claim 1, characterized in that R¹ represents a group selected from: 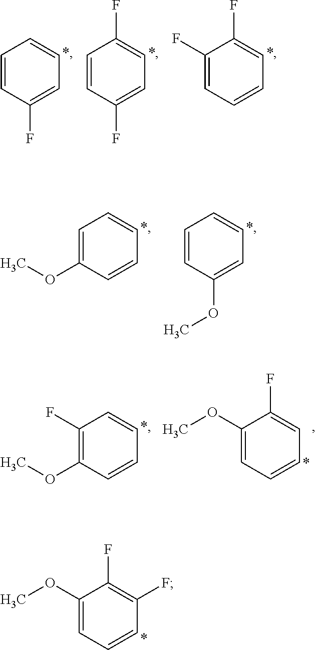
wherein * indicates the point of attachment of said groups to the rest of the molecule.

10. The method according to claim 1, characterized in that
R² represents 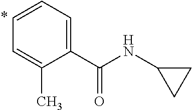
wherein * indicates the point of attachment of said group with the rest of the molecule.

11. The method according to claim 1, characterized in that
R^{3a} represents a group selected from: 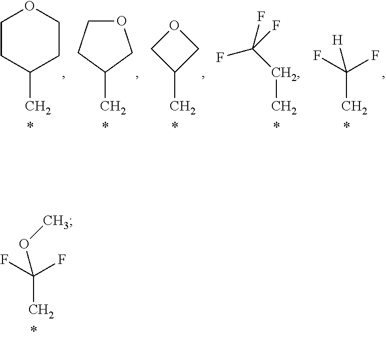
wherein * indicates the point of attachment of said groups with the rest of the molecule; and R^{3b} represents hydrogen atom.

12. The method according to claim 1, characterized in that
R²—Y represents

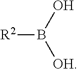

13. The method according to claim 1, comprising the following steps:
(a) allowing 8-bromo-6-chloro-3-iodoimidazo[1,2-b]pyridazine or 6,8-dibromo-3-iodoimidazo[1,2-b]pyridazine to react with 2,3-difluoro-4-methoxyphenol; thereby giving 6,8-bis(2,3-difluoro-4-methoxyphenoxy)-3-iodoimidazo[1,2-b]pyridazine;
(b) allowing 6,8-Bis(2,3-difluoro-4-methoxyphenoxy)-3-iodoimidazo[1,2-b]pyridazine to react with [4-(cyclopropylcarbamoyl)-3-methylphenyl]boronic acid; thereby giving 4-[6,8-bis(2,3-difluoro-4-methoxyphenoxy)imidazo[1,2-b]pyridazin-3-yl]-N-cyclopropyl-2-methylbenzamide;
(c) allowing 4-[6,8-bis(2,3-difluoro-4-methoxyphenoxy)imidazo[1,2-b]pyridazin-3-yl]-N-cyclopropyl-2-methylbenzamide to react with 3,3,3-trifluoropropan-1-amine; thereby giving N-Cyclopropyl-4-{6-(2,3-difluor-4-methoxyphenoxy)-8-[(3,3,3-trifluorpropyl)amino]imidazo[1,2-b]pyridazin-3-yl}-2-methylbenzamide.

14. The method according to claim 13, additionally comprising the following steps:
(d) adding the product N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide obtained in step (c) to water in order to precipitate the product;
(e) optionally drying the precipitated product obtained in step (d) in vacuum;
(f) suspending the precipitated product obtained in step (d) or (e) in toluene and heating the suspension to the boiling point of the suspension;
(g) cooling the suspension obtained in step (f) to a temperature below 50° C.; thereby obtaining N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide in crystalline form.

15. N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide prepared according to the method of claim 13.

16. N-cyclopropyl-4-{6-(2,3-difluor-4-methoxyphenoxy)-8-[(3,3,3-trifluorpropyl)amino]imidazo[1,2-b]pyridazin-3-yl}-2-methylbenzamide in crystalline form, characterized in that the x-ray diffractogram exhibits peak maxima of the 2 theta angle at about 3.7, 17.4, 21.3, and 23.9.

17. A compound of general formula (IV): 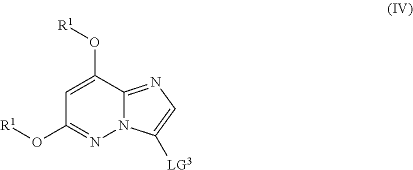
in which R¹ and LG³ are as defined in claim 1.

18. The compound according to claim 17 which is 6,8-bis(2,3-difluoro-4-methoxyphenoxy)-3-iodoimidazo[1,2-b]pyridazine.

19. A compound of general formula (VI): 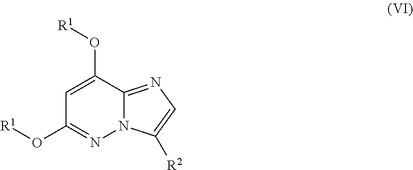
in which R¹ and R² are as defined in claim 1.

20. The compound according to claim 19 which is 4-[6,8-bis(2,3-difluoro-4-methoxyphenoxy)imidazo[1,2-b]pyridazin-3-yl]-N-cyclopropyl-2-methylbenzamide.

21. (canceled)
